SEQUENCE LISTING (106 sequences)

SEQ ID NO: 1 - protein, 25 aa, Artificial sequence
  Placental Immunoregulatory Ferritin (PLIF) derived polypeptide sequence
HHLLRPRRRKRPHSIPTPILIFRSP

SEQ ID NO: 2 - protein, 13 aa, Artificial sequence
  A monomer of the multimeric peptide
HSIPTPILIFRSP

SEQ ID NO: 3 - protein, 14 aa, Artificial sequence
  A monomer of the multimeric peptide
HLLRPRRRKRPHSI

SEQ ID NO: 4 - protein, 12 aa, Artificial sequence
  A monomer of the multimeric peptide
RPRRRKRPHSIP

SEQ ID NO: 5 - protein, 12 aa, Artificial sequence
  A monomer of the multimeric peptide
SIPTPILIFRSP

SEQ ID NO: 6 - protein, 14 aa, Artificial sequence
  A monomer of the multimeric peptide
PHSIPTPILIFRSP

SEQ ID NO: 7 - protein, 11 aa, Artificial sequence
  A monomer of the multimeric peptide
HHLLRPRRRKR

SEQ ID NO: 8 - protein, 15 aa, Artificial sequence
  Exemplary monomer of the multimeric peptide
CGHSIPTPILIFRSP

SEQ ID NO: 9 - protein, 16 aa, Artificial sequence
  Exemplary monomer of the multimeric peptide
CGHLLRPRRRKRPHSI

SEQ ID NO: 10 - protein, 14 aa, Artificial sequence
  Exemplary monomer of the multimeric peptide
CGRPRRRKRPHSIP

SEQ ID NO: 11 - protein, 14 aa, Artificial sequence
  Exemplary monomer of the multimeric peptide
CGSIPTPILIFRSP

SEQ ID NO: 12 - protein, 16 aa, Artificial sequence
  Exemplary monomer of the multimeric peptide
CGPHSIPTPILIFRSP

SEQ ID NO: 13 - protein, 13 aa, Artificial sequence
  Exemplary monomer of the multimeric peptide
CGHHLLRPRRRKR

SEQ ID NO: 14 - protein, 15 aa, Artificial sequence
  Placental Immunoregulatory Ferritin (PLIF) derived polypeptide sequence
RPHSIPTPILIFRSP

SEQ ID NO: 15 - protein, 6 aa, Artificial sequence
  Exemplary monomer of the multimeric peptide
HHLLRP

SEQ ID NO: 16 - protein, 6 aa, Artificial sequence
  Exemplary monomer of the multimeric peptide
HLLRPR

SEQ ID NO: 17 - protein, 6 aa, Artificial sequence
  Exemplary monomer of the multimeric peptide
LLRPRR

SEQ ID NO: 18 - protein, 6 aa, Artificial sequence
  Exemplary monomer of the multimeric peptide
LRPRRK

SEQ ID NO: 19 - protein, 6 aa, Artificial sequence
  Exemplary monomer of the multimeric peptide
RPRRKR

SEQ ID NO: 20 - protein, 6 aa, Artificial sequence
  Exemplary monomer of the multimeric peptide
PRRKRP

SEQ ID NO: 21 - protein, 6 aa, Artificial sequence
  Exemplary monomer of the multimeric peptide
RRKRPH

SEQ ID NO: 22 - protein, 6 aa, Artificial sequence
  Exemplary monomer of the multimeric peptide
RKRPHS

SEQ ID NO: 23 - protein, 6 aa, Artificial sequence
  Exemplary monomer of the multimeric peptide
KRPHSI

SEQ ID NO: 24 - protein, 6 aa, Artificial sequence
  Exemplary monomer of the multimeric peptide
RPHSIP

SEQ ID NO: 25 - protein, 6 aa, Artificial sequence
  Exemplary monomer of the multimeric peptide
PHSIPT

SEQ ID NO: 26 - protein, 6 aa, Artificial sequence
  Exemplary monomer of the multimeric peptide
HSIPTP

SEQ ID NO: 27 - protein, 6 aa, Artificial sequence
  Exemplary monomer of the multimeric peptide
SIPTPI

SEQ ID NO: 28 - protein, 6 aa, Artificial sequence
  Exemplary monomer of the multimeric peptide
IPTPIL

SEQ ID NO: 29 - protein, 6 aa, Artificial sequence
  Exemplary monomer of the multimeric peptide
PTPILI

SEQ ID NO: 30 - protein, 6 aa, Artificial sequence
  Exemplary monomer of the multimeric peptide
TPILIF

SEQ ID NO: 31 - protein, 6 aa, Artificial sequence
  Exemplary monomer of the multimeric peptide
PILIFR

SEQ ID NO: 32 - protein, 6 aa, Artificial sequence
  Exemplary monomer of the multimeric peptide
ILIFRS

SEQ ID NO: 33 - protein, 6 aa, Artificial sequence
  Exemplary monomer of the multimeric peptide
LIFRSP

SEQ ID NO: 34 - protein, 7 aa, Artificial sequence
  Exemplary monomer of the multimeric peptide
HHLLRPR

SEQ ID NO: 35 - protein, 7 aa, Artificial sequence
  Exemplary monomer of the multimeric peptide
HLLRPRR

SEQ ID NO: 36 - protein, 7 aa, Artificial sequence
  Exemplary monomer of the multimeric peptide
LLRPRRK

SEQ ID NO: 37 - protein, 7 aa, Artificial sequence
  Exemplary monomer of the multimeric peptide
LRPRRKR

SEQ ID NO: 38 - protein, 7 aa, Artificial sequence
  Exemplary monomer of the multimeric peptide
RPRRKRP

SEQ ID NO: 39 - protein, 7 aa, Artificial sequence
  Exemplary monomer of the multimeric peptide
PRRKRPH

SEQ ID NO: 40 - protein, 7 aa, Artificial sequence
  Exemplary monomer of the multimeric peptide
RRKRPHS

SEQ ID NO: 41 - protein, 7 aa, Artificial sequence
  Exemplary monomer of the multimeric peptide
RKRPHSI

SEQ ID NO: 42 - protein, 7 aa, Artificial sequence
  Exemplary monomer of the multimeric peptide
KRPHSIP

SEQ ID NO: 43 - protein, 7 aa, Artificial sequence
  Exemplary monomer of the multimeric peptide
RPHSIPT

SEQ ID NO: 44 - protein, 7 aa, Artificial sequence
  Exemplary monomer of the multimeric peptide
PHSIPTP

SEQ ID NO: 45 - protein, 7 aa, Artificial sequence
  Exemplary monomer of the multimeric peptide
HSIPTPI

SEQ ID NO: 46 - protein, 7 aa, Artificial sequence
  Exemplary monomer of the multimeric peptide
SIPTPIL

SEQ ID NO: 47 - protein, 7 aa, Artificial sequence
  Exemplary monomer of the multimeric peptide
IPTPILI

SEQ ID NO: 48 - protein, 7 aa, Artificial sequence
  Exemplary monomer of the multimeric peptide
PTPILIF

SEQ ID NO: 49 - protein, 7 aa, Artificial sequence
  Exemplary monomer of the multimeric peptide
TPILIFR

SEQ ID NO: 50 - protein, 7 aa, Artificial sequence
  Exemplary monomer of the multimeric peptide
PILIFRS

SEQ ID NO: 51 - protein, 7 aa, Artificial sequence
  Exemplary monomer of the multimeric peptide
ILIFRSP

SEQ ID NO: 52 - protein, 8 aa, Artificial sequence
  Exemplary monomer of the multimeric peptide
HHLLRPRR

SEQ ID NO: 53 - protein, 8 aa, Artificial sequence
  Exemplary monomer of the multimeric peptide
HLLRPRRK

SEQ ID NO: 54 - protein, 8 aa, Artificial sequence
  Exemplary monomer of the multimeric peptide
LLRPRRKR

SEQ ID NO: 55 - protein, 8 aa, Artificial sequence
  Exemplary monomer of the multimeric peptide
LRPRRKRP

SEQ ID NO: 56 - protein, 8 aa, Artificial sequence
  Exemplary monomer of the multimeric peptide
RPRRKRPH

SEQ ID NO: 57 - protein, 8 aa, Artificial sequence
  Exemplary monomer of the multimeric peptide
PRRKRPHS

SEQ ID NO: 58 - protein, 8 aa, Artificial sequence
  Exemplary monomer of the multimeric peptide
RRKRPHSI

SEQ ID NO: 59 - protein, 8 aa, Artificial sequence
  Exemplary monomer of the multimeric peptide
RKRPHSIP

SEQ ID NO: 60 - protein, 8 aa, Artificial sequence
  Exemplary monomer of the multimeric peptide
KRPHSIPT

SEQ ID NO: 61 - protein, 8 aa, Artificial sequence
  Exemplary monomer of the multimeric peptide
RPHSIPTP

SEQ ID NO: 62 - protein, 8 aa, Artificial sequence
  Exemplary monomer of the multimeric peptide
PHSIPTPI

SEQ ID NO: 63 - protein, 8 aa, Artificial sequence
  Exemplary monomer of the multimeric peptide
HSIPTPIL

SEQ ID NO: 64 - protein, 8 aa, Artificial sequence
  Exemplary monomer of the multimeric peptide
SIPTPILI

SEQ ID NO: 65 - protein, 8 aa, Artificial sequence
  Exemplary monomer of the multimeric peptide
IPTPILIF

SEQ ID NO: 66 - protein, 8 aa, Artificial sequence
  Exemplary monomer of the multimeric peptide
PTPILIFR

SEQ ID NO: 67 - protein, 8 aa, Artificial sequence
  Exemplary monomer of the multimeric peptide
TPILIFRS

SEQ ID NO: 68 - protein, 8 aa, Artificial sequence
  Exemplary monomer of the multimeric peptide
PILIFRSP

SEQ ID NO: 69 - protein, 9 aa, Artificial sequence
  Exemplary monomer of the multimeric peptide
HHLLRPRRK

SEQ ID NO: 70 - protein, 9 aa, Artificial sequence
  Exemplary monomer of the multimeric peptide
HLLRPRRKR

SEQ ID NO: 71 - protein, 9 aa, Artificial sequence
  Exemplary monomer of the multimeric peptide
LLRPRRKRP

SEQ ID NO: 72 - protein, 9 aa, Artificial sequence
  Exemplary monomer of the multimeric peptide
LRPRRKRPH

SEQ ID NO: 73 - protein, 9 aa, Artificial sequence
  Exemplary monomer of the multimeric peptide
RPRRKRPHS

SEQ ID NO: 74 - protein, 9 aa, Artificial sequence
  Exemplary monomer of the multimeric peptide
PRRKRPHSI

SEQ ID NO: 75 - protein, 9 aa, Artificial sequence
  Exemplary monomer of the multimeric peptide
RRKRPHSIP

SEQ ID NO: 76 - protein, 9 aa, Artificial sequence
  Exemplary monomer of the multimeric peptide
RKRPHSIPT

SEQ ID NO: 77 - protein, 9 aa, Artificial sequence
  Exemplary monomer of the multimeric peptide
KRPHSIPTP

SEQ ID NO: 78 - protein, 9 aa, Artificial sequence
  Exemplary monomer of the multimeric peptide
RPHSIPTPI

SEQ ID NO: 79 - protein, 9 aa, Artificial sequence
  Exemplary monomer of the multimeric peptide
PHSIPTPIL

SEQ ID NO: 80 - protein, 9 aa, Artificial sequence
  Exemplary monomer of the multimeric peptide
HSIPTPILI

SEQ ID NO: 81 - protein, 9 aa, Artificial sequence
  Exemplary monomer of the multimeric peptide
SIPTPILIF

SEQ ID NO: 82 - protein, 9 aa, Artificial sequence
  Exemplary monomer of the multimeric peptide
IPTPILIFR

SEQ ID NO: 83 - protein, 9 aa, Artificial sequence
  Exemplary monomer of the multimeric peptide
PTPILIFRS

SEQ ID NO: 84 - protein, 9 aa, Artificial sequence
  Exemplary monomer of the multimeric peptide
TPILIFRSP

SEQ ID NO: 85 - protein, 10 aa, Artificial sequence
  Exemplary monomer of the multimeric peptide
HHLLRPRRKR

SEQ ID NO: 86 - protein, 10 aa, Artificial sequence
  Exemplary monomer of the multimeric peptide
HLLRPRRKRP

SEQ ID NO: 87 - protein, 10 aa, Artificial sequence
  Exemplary monomer of the multimeric peptide
LLRPRRKRPH

SEQ ID NO: 88 - protein, 10 aa, Artificial sequence
  Exemplary monomer of the multimeric peptide
LRPRRKRPHS

SEQ ID NO: 89 - protein, 10 aa, Artificial sequence
  Exemplary monomer of the multimeric peptide
RPRRKRPHSI

SEQ ID NO: 90 - protein, 10 aa, Artificial sequence
  Exemplary monomer of the multimeric peptide
PRRKRPHSIP

SEQ ID NO: 91 - protein, 10 aa, Artificial sequence
  Exemplary monomer of the multimeric peptide
RRKRPHSIPT

SEQ ID NO: 92 - protein, 10 aa, Artificial sequence
  Exemplary monomer of the multimeric peptide
RKRPHSIPTP

SEQ ID NO: 93 - protein, 10 aa, Artificial sequence
  Exemplary monomer of the multimeric peptide
KRPHSIPTPI

SEQ ID NO: 94 - protein, 10 aa, Artificial sequence
  Exemplary monomer of the multimeric peptide
RPHSIPTPIL

SEQ ID NO: 95 - protein, 10 aa, Artificial sequence
  Exemplary monomer of the multimeric peptide
PHSIPTPILI

SEQ ID NO: 96 - protein, 10 aa, Artificial sequence
  Exemplary monomer of the multimeric peptide
HSIPTPILIF

SEQ ID NO: 97 - protein, 10 aa, Artificial sequence
  Exemplary monomer of the multimeric peptide
SIPTPILIFR

SEQ ID NO: 98 - protein, 10 aa, Artificial sequence
  Exemplary monomer of the multimeric peptide
IPTPILIFRS

SEQ ID NO: 99 - protein, 10 aa, Artificial sequence
  Exemplary monomer of the multimeric peptide
PTPILIFRSP

SEQ ID NO: 100 - protein, 48 aa, Artificial sequence
  Placental Immunoregulatory Ferritin (PLIF), C′, derived peptide C48
FPSPISPSPSCWHHYTTNRPQPQHHLLRPRRRKRPHSIPTPILIFRSP

SEQ ID NO: 101 - protein, 27 aa, Artificial sequence
  multimeric synthetic peptide
CGHHLLRPRRRKRPHSIPTPILIFRSP

SEQ ID NO: 102 - protein, 27 aa, Artificial Sequence
  multimeric synthetic peptide
  DISULFID (1)..(1): polypeptide dimerizes through disulfide bond on cys number one
CGHHLLRPRRRKRPHSIPTPILIFRSP

SEQ ID NO: 103 - protein, 509 aa, Homo sapiens
MGCGCSSHPEDDWMENIDVCENCHYPIVPLDGKGTLLIRNGSEVRDPLVTYEGSNPPASPLQDNLVIALHSYEPSHDGDLGFEKGEQLRILEQSGEWWKAQSLTTGQEGFIPFNFVAKANSLEPEPWFFKNLSRKDAERQLLAPGNTHGSFLIRESESTAGSFSLSVRDFDQNQGEVVKHYKIRNLDNGGFYISPRITFPGLHELVRHYTNASDGLCTRLSRPCQTQKPQKPWWEDEWEVPRETLKLVERLGAGQFGEVWMGYYNGHTKVAVKSLKQGSMSPDAFLAEANLMKQLQHQRLVRLYAVVTQEPIYIITEYMENGSLVDFLKTPSGIKLTINKLLDMAAQIAEGMAFIEERNYIHRDLRAANILVSDTLSCKIADFGLARLIEDNEYTAREGAKFPIKWTAPEAINYGTFTIKSDVWSFGILLTEIVTHGRIPYPGMTNPEVIQNLERGYRMVRPDNCPEELYQLMRLCWKERPEDRPTFDYLRSVLEDFFTATEGQYQPQP

SEQ ID NO: 104 - protein, 619 aa, Homo sapiens
MPDPAAHLPFFYGSISRAEAEEHLKLAGMADGLFLLRQCLRSLGGYVLSLVHDVRFHHFPIERQLNGTYAIAGGKAHCGPAELCEFYSRDPDGLPCNLRKPCNRPSGLEPQPGVFDCLRDAMVRDYVRQTWKLEGEALEQAIISQAPQVEKLIATTAHERMPWYHSSLTREEAERKLYSGAQTDGKFLLRPRKEQGTYALSLIYGKTVYHYLISQDKAGKYCIPEGTKFDTLWQLVEYLKLKADGLIYCLKEACPNSSASNASGAAAPTLPAHPSTLTHPQRRIDTLNSDGYTPEPARITSPDKPRPMPMDTSVYESPYSDPEELKDKKLFLKRDNLLIADIELGCGNFGSVRQGVYRMRKKQIDVAIKVLKQGTEKADTEEMMREAQIMHQLDNPYIVRLIGVCQAEALMLVMEMAGGGPLHKFLVGKREEIPVSNVAELLHQVSMGMKYLEEKNFVHRDLAARNVLLVNRHYAKISDFGLSKALGADDSYYTARSAGKWPLKWYAPECINFRKFSSRSDVWSYGVTMWEALSYGQKPYKKMKGPEVMAFIEQGKRMECPPECPPELYALMSDCWIYKWEDRPDFLTVEQRMRACYYSLASKVEGPPGSTQKAEAACA

SEQ ID NO: 105 - protein, 845 aa, Homo sapiens
MELWRQCTHWLIQCRVLPPSHRVTWDGAQVCELAQALRDGVLLCQLLNNLLPHAINLREVNLRPQMSQFLCLKNIRTFLSTCCEKFGLKRSELFEAFDLFDVQDFGKVIYTLSALSWTPIAQNRGIMPFPTEEESVGDEDIYSGLSDQIDDTVEEDEDLYDCVENEEAEGDEIYEDLMRSEPVSMPPKMTEYDKRCCCLREIQQTEEKYTDTLGSIQQHFLKPLQRFLKPQDIEIIFINIEDLLRVHTHFLKEMKEALGTPGAANLYQVFIKYKERFLVYGRYCSQVESASKHLDRVAAAREDVQMKLEECSQRANNGRFTLRDLLMVPMQRVLKYHLLLQELVKHTQEAMEKENLRLALDAMRDLAQCVNEVKRDNETLRQITNFQLSIENLDQSLAHYGRPKIDGELKITSVERRSKMDRYAFLLDKALLICKRRGDSYDLKDFVNLHSFQVRDDSSGDRDNKKWSHMFLLIEDQGAQGYELFFKTRELKKKWMEQFEMAISNIYPENATANGHDFQMFSFEETTSCKACQMLLRGTFYQGYRCHRCRASAHKECLGRVPPCGRHGQDFPGTMKKDKLHRRAQDKKRNELGLPKMEVFQEYYGLPPPPGAIGPFLRLNPGDIVELTKAEAEQNWWEGRNTSTNEIGWFPCNRVKPYVHGPPQDLSVHLWYAGPMERAGAESILANRSDGTFLVRQRVKDAAEFAISIKYNVEVKHIKIMTAEGLYRITEKKAFRGLTELVEFYQQNSLKDCFKSLDTTLQFPFKEPEKRTISRPAVGSTKYFGTAKARYDFCARDRSELSLKEGDIIKILNKKGQQGWWRGEIYGRVGWFPANYVEEDYSEYC

SEQ ID NO: 106 - protein, 1306 aa, Homo sapiens
MTMYLWLKLLAFGFAFLDTEVFVTGQSPTPSPTGLTTAKMPSVPLSSDPLPTHTTAFSPASTFERENDFSETTTSLSPDNTSTQVSPDSLDNASAFNTTGVSSVQTPHLPTHADSQTPSAGTDTQTFSGSAANAKLNPTPGSNAISDVPGERSTASTFPTDPVSPLTTTLSLAHHSSAALPARTSNTTITANTSDAYLNASETTTLSPSGSAVISTTTIATTPSKPTCDEKYANITVDYLYNKETKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDLHKKRSCNLDEQQELVERDDEKQLMNVEPIHADILLETYKRKIADEGRLFLAEFQSIPRVFSKFPIKEARKPFNQNKNRYVDILPYDYNRVELSEINGDAGSNYINASYIDGFKEPRKYIAAQGPRDETVDDFWRMIWEQKATVIVMVTRCEEGNRNKCAEYWPSMEEGTRAFGDVVVKINQHKRCPDYIIQKLNIVNKKEKATGREVTHIQFTSWPDHGVPEDPHLLLKLRRRVNAFSNFFSGPIVVHCSAGVGRTGTYIGIDAMLEGLEAENKVDVYGYVVKLRRQRCLMVQVEAQYILIHQALVEYNQFGETEVNLSELHPYLHNMKKRDPPSEPSPLEAEFQRLPSYRSWRTQHIGNQEENKSKNRNSNVIPYDYNRVPLKHELEMSKESEHDSDESSDDDSDSEEPSKYINASFIMSYWKPEVMIAAQGPLKETIGDFWQMIFQRKVKVIVMLTELKHGDQEICAQYWGEGKQTYGDIEVDLKDTDKSSTYTLRVFELRHSKRKDSRTVYQYQYTNWSVEQLPAEPKELISMIQVVKQKLPQKNSSEGNKHHKSTPLLIHCRDGSQQTGIFCALLNLLESAETEEVVDIFQVVKALRKARPGMVSTFEQYQFLYDVIASTYPAQNGQVKKNNHQEDKIEFDNEVDKVKQDANCVNPLGAPEKLPEAKEQAEGSEPTSGTEGPEHSVNGPASPALNQGS

(19) United States
(12) Patent Application Publication
(10) Pub. No.: US 2022/0120732 A1
(43) Pub. Date: Apr. 21, 2022

(54) TREATMENT OF DISEASES WITH MULTIMERIC PEPTIDES

(71) Applicant: Ramot at Tel-Aviv University Ltd., Tel-Aviv (IL)

(72) Inventor: Annat RAITER, Tel-Aviv (IL)

(73) Assignee: Ramot at Tel-Aviv University Ltd., Tel-Aviv (IL)

(21) Appl. No.: 17/428,714

(22) PCT Filed: Feb. 21, 2020
(86) PCT No.: PCT/IL2020/050193

§ 371 Date: Aug. 5, 2021

Related U.S. Application Data
(60) Provisional application No. 62/904,708, filed on Sep. 24, 2019.
(60) Provisional application No. 62/808,319, filed on Feb. 21, 2019.
(60) Provisional application No. 62/808,307, filed on Feb. 21, 2019.

(51) Int. Cl. G01N 33/50; A61P 35/00; C12N 5/071; C12N 5/09; C07K 14/47

(52) U.S. Cl. CPC G01N 33/505; A61P 35/00; C12N 5/0631; C12N 2501/24; C07K 14/47; G01N 2333/70589; C12N 2502/1114; C12N 5/0693

(57) ABSTRACT

Methods of treating diseases selected from the group consisting of an autoimmune disease, a neurodegenerative disease, triple negative breast cancer, head and neck cancer and an infectious disease are disclosed. The method comprises administering agents that bind to CD45.

### RELATED APPLICATIONS

This application claims the benefit of priority of U.S. Provisional Patent Application No. 62/808,307 filed 21 Feb. 2019, U.S. Provisional Patent Application No. 62/808,319 filed 21 Feb. 2019 and U.S. Provisional Patent Application No. 62/904,708 filed 24 Sep. 2019, the contents of which are incorporated herein by reference in their entirety.

### SEQUENCE LISTING STATEMENT

The ASCII file, entitled 81658 Sequence Listing.txt, created on 21 Feb. 2020, comprising 51,470 bytes, submitted concurrently with the filing of this application is incorporated herein by reference.

### FIELD AND BACKGROUND OF THE INVENTION

The present invention, in some embodiments thereof, relates to treatment of diseases associated with CD45 (a receptor-linked protein tyrosine phosphatase that is expressed on leucocytes) with multimeric peptides.

More than 7000 naturally occurring peptides have been identified, and these often have crucial roles in human physiology, including actions as hormones, neurotransmitters, growth factors, ion channel ligands, or anti-infectives. In general, peptides are selective and efficacious signaling molecules that bind to specific cell surface receptors, such as G protein-coupled receptors (GPCRs) or ion channels, where they trigger intracellular effects. Given their attractive pharmacological profile and intrinsic properties, peptides represent an excellent starting point for the design of novel therapeutics and their specificity has been seen to translate into excellent safety, tolerability, and efficacy profiles in humans. This aspect might also be the primary differentiating factor of peptides compared with traditional small molecules. Furthermore, peptide therapeutics are typically associated with lower production complexity compared with protein-based biopharmaceuticals and, therefore, the production costs are also lower, generally approaching those of small molecules. Thus, in several ways, peptides are in the sweet spot between small molecules and biopharmaceuticals.

Naturally occurring peptides are often not directly suitable for use as convenient therapeutics because they have intrinsic weaknesses, including poor chemical and physical stability, and a short circulating plasma half-life. These aspects must be addressed for their use as medicines.

Synthetic dimeric peptides are disclosed in WO2013/140389 for the treatment of cancer.

Additional background art includes U.S. Pat. No. 4,882,270 which discloses a method for detecting breast cancer, by using antibodies against isoferritin placental protein.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### SUMMARY OF THE INVENTION

According to one aspect of the present invention there is provided a method of treating a disease selected from the group consisting of an autoimmune disease, a neurodegenerative disease and an infectious disease in a subject in need thereof, the method comprising administering to the subject a therapeutically effective amount of a multimeric peptide comprising at least two peptide monomers linked to one another, each of the at least two peptide monomers comprising at least 6 consecutive amino acids from the amino acid sequence as set forth in SEQ ID NO: 1, wherein the at least two peptide monomers are each no longer than 30 amino acids, wherein the multimeric peptide binds to Receptor type tyrosine-protein phosphatase C (CD45), with the proviso that the infectious disease is not a retrovirally-mediated disease, thereby treating the disease.

According to one aspect of the present invention there is provided a multimeric peptide for use in treating a disease selected from the group consisting of an autoimmune disease, a neurodegenerative disease and an infectious disease, the multimeric peptide comprising at least two peptide monomers linked to one another, each of the at least two peptide monomers comprising at least 6 consecutive amino acids from the amino acid sequence as set forth in SEQ ID NO: 1, wherein the at least two peptide monomers are each no longer than 30 amino acids, wherein the multimeric peptide binds to CD45, with the proviso that the infectious disease is not a retrovirally-mediated disease.

According to some embodiments of the invention, the method or multimeric peptide of claim 1 or 2, wherein the peptide is capable of increasing INF-γ secretion from activated leukocytes.

According to some embodiments of the invention, the peptide is a dimer.

According to some embodiments of the invention, each of the at least two peptide monomers comprise no more than 15 consecutive amino acids from the amino acid sequence as set forth in SEQ ID NO: 1.

According to some embodiments of the invention, the multimeric peptide consists of the sequence as set forth in SEQ ID NO: 102.

According to some embodiments of the invention, the at least two peptide monomers comprise an identical amino acid sequence.

According to some embodiments of the invention, each of the at least two peptide monomers is attached to a Cysteine (Cys) residue.

According to some embodiments of the invention, the carboxy end of the at least two peptide monomers is attached to the Cys residue.

According to some embodiments of the invention, each of the two peptide monomers are attached via a non-peptide linker.

According to some embodiments of the invention, the at least two peptide monomers are linked to one another by a disulfide bond.

According to some embodiments of the invention, the disulfide bond is an intermolecular disulfide bond formed between the Cys residues.

According to some embodiments of the invention, the multimeric peptide further comprises a Gly residue connecting the Cys residue to the carboxy end of the at least two peptide monomers.

According to some embodiments of the invention, each of the two at least two peptide monomers comprise the sequence selected from the group consisting of SEQ ID NOs: 2-7.

According to some embodiments of the invention, each of the at least two peptide monomers consists of the sequence selected from the group consisting of SEQ ID NOs: 8-13 and 101.

According to some embodiments of the invention, the multimeric peptide comprises at least one synthetic amino acid.

According to some embodiments of the invention, the at least two peptide monomers are covalently linked to one another.

According to some embodiments of the invention, the disease is a neurodegenerative disease.

According to some embodiments of the invention, the viral disease is not hepatitis or HHV6.

According to some embodiments of the invention, the infectious disease is a bacterial disease.

According to some embodiments of the invention, the infectious disease is a fungal disease.

According to some embodiments of the invention, the autoimmune disease is systemic lupus erythematosus or graft versus host disease.

According to some embodiments of the invention, the disease is not chronic fatigue syndrome.

According to one aspect of the present invention there is provided an in-vitro method of activating T cells, the method comprising incubating T cells with pathogenic cells in the presence of an agent that binds to CD45 of the T cells, under conditions which allow expansion of the T cells, with the proviso that the agent is not a multimeric peptide comprising at least two peptide monomers linked to one another, each of the at least two peptide monomers comprising at least 6 consecutive amino acids from the amino acid sequence as set forth in SEQ ID NO: 1, wherein the at least two peptide monomers are each no longer than 30 amino acids.

According to one aspect of the present invention there is provided an in vitro method of increasing the cytotoxicity of T cells comprising incubating pathogenic cells with T cells in the presence of an agent that binds to CD45 of the T cells, under conditions which allow for the generation of activated T cells that are cytotoxic to the pathogenic cells, thereby increasing the cytotoxicity of the T cells, with the proviso that the agent is not a multimeric peptide comprising at least two peptide monomers linked to one another, each of the at least two peptide monomers comprising at least 6 consecutive amino acids from the amino acid sequence as set forth in SEQ ID NO: 1, wherein the at least two peptide monomers are each no longer than 30 amino acids.

According to some embodiments of the invention, the method further comprises expanding the activated T cells.

According to one aspect of the present invention there is provided an in vitro method of generating a cytotoxic T cell line comprising:

(a) incubating pathogenic cells with T cells in the presence of an agent which binds to CD45 under conditions which allow for the generation of activated T cells that are cytotoxic to the pathogenic cells; and

(b) expanding the activated T cells, thereby generating the cytotoxic T cell line, with the proviso that the agent is not a multimeric peptide comprising at least two peptide monomers linked to one another, each of the at least two peptide monomers comprising at least 6 consecutive amino acids from the amino acid sequence as set forth in SEQ ID NO: 1, wherein the at least two peptide monomers are each no longer than 30 amino acids.

According to some embodiments of the invention, the expanding is effected using interleukin 2 (IL-2).

According to some embodiments of the invention, the pathogenic cells have an upregulated amount of Placenta Immunomodulatory Factor (PLIF) as compared to healthy cells.

According to some embodiments of the invention, the pathogenic cells comprise cancer cells.

According to some embodiments of the invention, the cells comprise breast cancer cells.

According to some embodiments of the invention, the cancer cells comprise head and neck cancer cells.

According to some embodiments of the invention, the breast cancer cells comprise cells of the T47D or MCF-7 cell lines.

According to some embodiments of the invention, the T cells are comprised in peripheral mononuclear blood cells (PBMCs).

According to some embodiments of the invention, the agent is an antibody that binds to CD45.

According to some embodiments of the invention, the antibody is an inhibitory antibody.

According to one aspect of the present invention there is provided a method of treating triple negative breast cancer or head and neck cancer in a subject in need thereof, the method comprising administering to the subject a therapeutically effective amount of an agent that binds to Receptor type tyrosine-protein phosphatase C (CD45), with the proviso that the infectious disease is not a retrovirally-mediated disease, thereby treating the triple negative breast cancer or the head and neck cancer.

According to one aspect of the present invention there is provided an agent that binds to CD45 for use in treating triple negative breast cancer or head and neck cancer.

According to some embodiments of the invention, the agent is a peptide.

According to some embodiments of the invention, the disease is a viral disease.

According to some embodiments of the invention, the peptide is a multimeric peptide comprising at least two peptide monomers linked to one another, each of the at least two peptide monomers comprising at least 6 consecutive amino acids from the amino acid sequence as set forth in SEQ ID NO: 1, wherein the at least two peptide monomers are each no longer than 30 amino acids.

According to some embodiments of the invention, the agent is capable of increasing INF-γ secretion from activated leukocytes.

According to some embodiments of the invention, the peptide is a dimer.

According to some embodiments of the invention, each of the at least two peptide monomers comprise no more than 15 consecutive amino acids from the amino acid sequence as set forth in SEQ ID NO: 1.

According to some embodiments of the invention, each of the two at least two peptide monomers comprise the sequence selected from the group consisting of SEQ ID NOs: 2-7.

According to some embodiments of the invention, each of the at least two peptide monomers consists of the sequence selected from the group consisting of SEQ ID NOs: 8-13 and 101.

According to some embodiments of the invention, the at least two peptide monomers are covalently linked to one another.

According to another aspect of the present invention there is provided method of monitoring the efficacy of a therapeutic agent that increases the cytotoxicity of T cells by binding to CD45 in a subject, the method comprising analyzing in the T cells of the subject the phosphorylation status of at least one protein selected from the group consisting of Lck, ZAP70 and VAV-1, wherein:

(i) a decrease in the phosphorylation status of lymphocyte-specific protein tyrosine kinase (Lck) at position 505 is indicative of an efficacious therapeutic agent;

(ii) an increase in the phosphorylation status of Lck at position 394 is indicative of an efficacious therapeutic agent;

(iii) an increase in the phosphorylation status of Vav Guanine Nucleotide Exchange Factor 1 (VAV-1) is indicative of an efficacious therapeutic agent; and/or

(iv) an increase in the phosphorylation status of Zeta-chain-associated protein kinase 70 (ZAP-70) at position 493 is indicative of an efficacious therapeutic agent.

According to embodiments of the present invention, the agent is a multimeric peptide comprising at least two peptide monomers linked to one another, each of said at least two peptide monomers comprising at least 6 consecutive amino acids from the amino acid sequence as set forth in SEQ ID NO: 1, wherein said at least two peptide monomers are each no longer than 30 amino acids.

### BRIEF DESCRIPTION OF THE SEVERAL VIEWS OF THE DRAWINGS

Some embodiments of the invention are herein described, by way of example only, with reference to the accompanying images. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 provides the structure and amino acid sequence of the C24D (an exemplary peptide according to embodiments of the present invention)—SEQ ID NO: 102.

FIG. 2 is a series of photographs illustrating the cytotoxic activity of the C24D peptide in the following breast cancer cell lines: MCF7, MDAMB231 and MDAMB468.

FIGS. 3A-B are photographs illustrating patient derived tumor cells in the presence (FIG. 3B) and the absence (FIG. 3A) of autologous PBMCs.

FIGS. 4A-B are photographs illustrating patient derived tumor cells in the presence (FIG. 4B) and the absence (FIG. 4A) of autologous PBMCs+C24D.

FIGS. 5A-B are photographs illustrating patient derived stromal cells in the presence (FIG. 5B) and the absence (FIG. 5A) of autologous PBMCs+C24D.

FIG. 6 is a bar graph illustrating the apoptotic effect of the C24D peptide on breast cancer cells.

FIG. 7 is a representative FACs analysis illustrating the apoptotic effect of the C24D peptide on breast cancer cells.

FIGS. 8A-C are graphs illustrating that the C24D peptide induces interferon secretion in breast cancer cells.

FIG. 9 is a bar graph illustrating that the C24D peptide binds to different PBMC subpopulations.

FIG. 10 is a bar graph illustrating the extent of C24D binding in MCF7 cells.

FIG. 11 is a bar graph illustrating the extent of C24D binding in MDAMB468 cells.

FIG. 12 is a bar graph illustrating the extent of C24D binding in MDAMB231 cells.

FIG. 13 is a photograph of a protein gel portraying the unique cell surface receptor found in the two PBMC samples and not found in the control samples.

FIGS. 14A-C are photographs illustrating that C24D triggers immune killing of Head & Neck cancer cells.

FIG. 15 is a bar graph illustrating that C24D induces interferon gamma secretion in Head & Neck cancer cells.

FIG. 16 is a bar graph illustrating that C24D activates PBMCs in Head & Neck cancer cells.

FIG. 17 are graphs illustrating that C24D reverses tumor suppression by re-activation of Src kinase signaling in PBMCs.

FIG. 18 are graphs illustrating that C24D induced Src kinases signaling in PBMC of metastatic breast cancer patients.

FIG. 19 are graphs illustrating that C24D immune system re-activation is specific and occurs only in the presence of tumors.

FIG. 20 is a graph illustrating that C24D binds to the CD45 receptor on leukocytes.

FIGS. 21A-B illustrates the results of Western blot analysis of PBMCs co-cultured with triple negative breast cancer (TNBC) cells and treated with C24D.

FIG. 22 is a cartoon illustrating the mechanism of tumor escape which is reversed by treatment with C24D. In the deactivated state, Lck Tyr 505 is phosphorylated and Tyr 394 is de-phosphorylated. The Tyr 493 in ZAP70 is de-phosphorylated as well as VAV-1. This leads to immune cell suppression. In the activated state, addition of C24D reverses tumor immune suppression by binding to CD45. This induces dephosphorylation of the Lck's inhibitory Tyr505 and phosphorylation of Tyr394, resulting in VAV-1 and ZAP-70 phosphorylation and activation.

### DESCRIPTION OF SPECIFIC EMBODIMENTS OF THE INVENTION

The present invention, in some embodiments thereof, relates to treatment of diseases associated with CD45 (a receptor-linked protein tyrosine phosphatase that is expressed on leucocytes) with multimeric peptides.

Cancer cells display multiple immunosuppressive mechanisms to evade T-cell responses. Malignant cells can escape immune elimination through loss of antigenicity and/or loss of immunogenicity and by managing an immunosuppressive microenvironment. This tumor microenvironment is conditional to tumor heterogeneity affecting the ability of the immune system to control the tumor. During the last decade this inter-cellular crosstalk between tumor cells and immune cells has resulted in the development of novel immunotherapeutic strategies in order to restrain the mechanisms leading to escape of tumor cells from immune surveillance. Different monoclonal antibodies directed against immune checkpoints, e.g., the programmed cell death protein 1/programmed cell death ligand 1 (PD1/PDL-1) with or without the combination with cytotoxic T-lymphocyte-associated antigen 4 (CTLA-4) have been successfully implemented for the treatment of some cancers. Despite promising results obtained in some solid and hematologic tumor types, not all tumors and patients respond to these immunomodulatory therapies. Although the immune system can be harnessed with these new therapeutic strategies, some clinically relevant tumors establish a microenvironment that suppresses productive anti-tumor immunity.

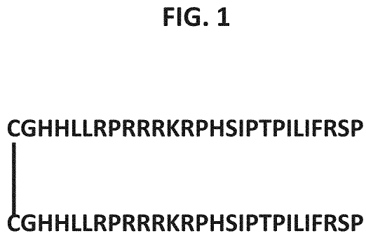

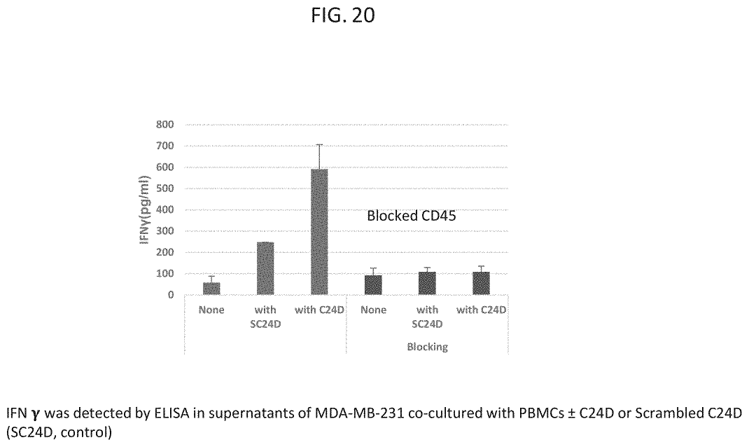

The present inventors have previously identified a multimeric peptide (referred to herein as C24D—see FIG. 1) which enables leukocytes to specifically kill tumor cells. The present inventors have now discovered that the mechanism of action behind C24D's cytotoxic activity is its binding to the CD45 receptor on T and natural killer (NK) cells (see FIG. 20). By binding to CD45 on immunocompetent cells, C24D breaks cancer-cell-induced Src tyrosine kinase inhibition, resulting in an anti-tumor response. This mechanism differentiates C24D from other recently developed cancer immunotherapies, suggesting an effective therapeutic against breast cancer and other unresponsive cancers.

The present inventors thus propose that C24D may be useful for treating diseases in addition to cancer which are mediated by the activity of the CD45 receptor.

Such diseases include autoimmune diseases, neurodegenerative diseases and infectious diseases.

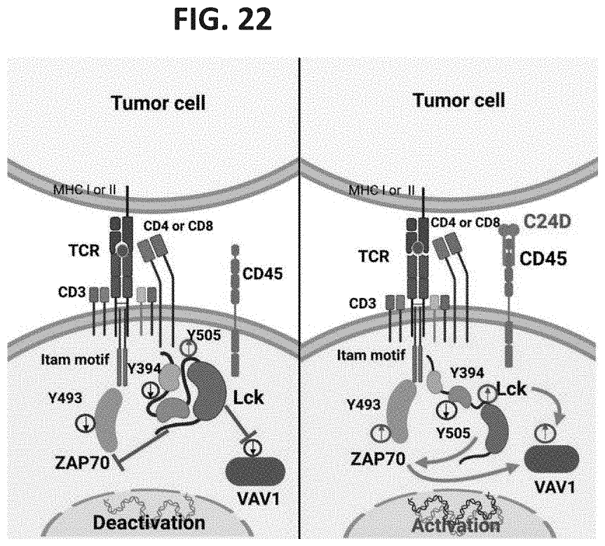

Whilst further reducing the present invention to practice, the present inventors have shown that C24D binding to CD45 reverses tumor suppression in CD8+ cells, CD4+ cells and NK cells, by phosphorylation of Lck 394, ZAP70 Y493, VAV1 and de-phosphorylation of Lck Y505 in leukocytes, resulting in TCR activation and immune-modulated tumor cell killing—see FIG. 22. Consequently, the present teachings suggest that the phosphorylation status of any of the above mentioned proteins can be used to assess the therapeutic efficacy of the presently disclosed agents.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

According to an aspect of the present invention there is provided a method of treating a disease selected from the group consisting of an autoimmune disease, a neurodegenerative disease and an infectious disease in a subject in need thereof, the method comprising administering to the subject a therapeutically effective amount of a multimeric peptide comprising at least two peptide monomers linked to one another, each of said at least two peptide monomers comprising at least 6 consecutive amino acids from the amino acid sequence as set forth in SEQ ID NO: 1, wherein said at least two peptide monomers are each no longer than 30 amino acids, wherein said multimeric peptide binds to Receptor type tyrosine-protein phosphatase C (CD45), with the proviso that the infectious disease is not a retrovirally-mediated disease, thereby treating the disease.

The phrase “multimeric peptide” as used herein, describes a peptide formed from two or more peptide monomers (i.e. two or more peptide chains) that are associated covalently or non-covalently, with or without linkers. It will be appreciated that the peptide monomers are not linked together so as to form an amide bond through the amine group of one monomer and the carboxylic acid group of the other monomer so as to form a single extended chain.

According to a particular embodiment, the multimeric peptide is a dimer (i.e. comprises two peptide monomers that are associated covalently or non-covalently, with or without linkers). According to a particular embodiment, the two peptide monomers are not linked via a peptide bond.

The multimeric peptides disclosed herein are capable of binding to CD45.

As used herein, the term “CD45” refers to the protein encoded by the PTPRC gene. It is a member of the protein tyrosine phosphatase (PTP) family. PTPs are known to be signaling molecules that regulate a variety of cellular processes including cell growth, differentiation, mitosis, and oncogenic transformation. CD45 contains an extracellular domain, a single transmembrane segment and two tandem intracytoplasmic catalytic domains, and thus is classified as a receptor type PTP. CD45 has been shown to be an essential regulator of T- and B-cell antigen receptor signaling. It functions through either direct interaction with components of the antigen receptor complexes, or by activating various Src family kinases required for the antigen receptor signaling. CD45 also suppresses JAK kinases, and thus functions as a regulator of cytokine receptor signaling.

RefSeq numbers of CD45 include PTPRC_Human, P08575 gene, HGNC (9666), Entrez Gene (5788), Ensembl (ENSG00000081237), OMIM (151460), UniProtKB (P08575). GeneBank AA403163 AA904360 AK130573 AK2921131 AK299986. RefSeq NM_001267798 NM_002838 NM_080921 NM_080922.

An exemplary sequence of human CD45 is set forth in SEQ ID NO:103.

Methods of analyzing whether peptides are capable of binding to CD45 include Peptide array, Phage display peptide libraries, Mass spectrometry, Reverse Phase Protein Arrays, Yeast Two-Hybrid, Plate-based and biophysical assays such as Fluorescence anisotropy or fluorescence polarization which is widely used to measure the binding of labeled peptide ligands to domains. X-ray crystallography can be used to locate the exact position of epitope within the protein structure.

In one embodiment, the multimeric peptides disclosed herein are capable of blocking binding of PLIF to its receptor on white blood cells, thereby acting as an antagonist to the endogenous activity of Placenta Immunomodulatory Factor (PLIF).

PLIF is a protein composed of 165 amino acids. Of these, 117 match the ferritin heavy chain sequence, whereas the C-terminal 48 amino acids (C48) has a sequence which is not related to ferritin—SEQ ID NO: 100. It has been shown that the subcloned recombinant C48 peptide exhibits the bioactivity and therapeutic properties of PLIF [Moroz et al, J. Biol. Chem. 2002, 277, 12901-12905].

Binding affinity can be measured by any assay known or available to those skilled in the art, including but not limited to BIAcore measurements, ELISA assays, competition assays, etc. Bioactivity can be measured in vivo or in vitro by any assay known or available to those skilled in the art.

The multimeric peptides of this aspect of the present invention typically comprise additional functions such as being capable of increasing interferon gamma (INF-γ) secretion and/or reduction of secretion of interleukin-10 (IL-10) from activated leukocytes.

According to one embodiment, secretion of INF-γ is increased by at least two fold, or more preferably by at least five fold the amount of INF-γ that is basally secreted from activated leukocytes (i.e. in the absence of the disclosed peptides).

Methods of analyzing INF-γ secretion include but are not limited to ELISA kits such as those available from DPC, and R&D Systems, USA.

In some embodiments, the multimeric peptide is such that the amino acid sequence of each of its monomers are the same, thus forming a homomultimeric peptide. When the multimeric peptide is a dimer and the two monomers are identical, a homodimeric peptide is formed.

In some embodiments, the multimeric peptide is such that the amino acid sequence of at least two of its peptide monomers are different, thus forming a heteromultimeric peptide. When the multimeric peptide is a dimer and the two monomers are different, a heterodimeric peptide is formed.

As mentioned, the monomers of the multimeric peptide of this aspect of the present invention are derived from the C terminal amino acids of Placenta Immunomodulatory Factor (PLIF). In a particular embodiment, the peptides include at least 6 consecutive amino acids from the sequence as set forth in SEQ ID NO: 1 (His-His-Leu-Leu-Arg-Pro-Arg-Arg-Lys-Arg-Pro-His-Ser-Ile-Pro-Thr-Pro-Ile-Leu-Ile-Phe-Arg-Ser-Pro).

According to some embodiments, each monomer of the multimeric peptide comprises at least 7 consecutive amino acids from the sequence as set forth in SEQ ID NO: 1.

According to some embodiments, each monomer of the multimeric peptide comprises at least 8 consecutive amino acids from the sequence as set forth in SEQ ID NO: 1.

According to some embodiments, each monomer of the multimeric peptide comprises at least 9 consecutive amino acids from the sequence as set forth in SEQ ID NO: 1.

According to some embodiments, each monomer of the multimeric peptide comprises at least 10 consecutive amino acids from the sequence as set forth in SEQ ID NO: 1.

According to some embodiments, each monomer of the multimeric peptide comprises at least 11 consecutive amino acids from the sequence as set forth in SEQ ID NO: 1.

According to some embodiments, each monomer of the multimeric peptide comprises at least 12 consecutive amino acids from the sequence as set forth in SEQ ID NO: 1.

According to some embodiments, each monomer of the multimeric peptide comprises at least 13 consecutive amino acids from the sequence as set forth in SEQ ID NO: 1.

According to some embodiments, each monomer of the multimeric peptide comprises at least 14 consecutive amino acids from the sequence as set forth in SEQ ID NO: 1.

According to some embodiments, each monomer of the multimeric peptide comprises at least 15 consecutive amino acids from the sequence as set forth in SEQ ID NO: 1.

According to some embodiments, each monomer of the multimeric peptide comprises at least 16 consecutive amino acids from the sequence as set forth in SEQ ID NO: 1.

According to some embodiments, each monomer of the multimeric peptide comprises at least 17 consecutive amino acids from the sequence as set forth in SEQ ID NO: 1.

According to some embodiments, each monomer of the multimeric peptide comprises at least 18 consecutive amino acids from the sequence as set forth in SEQ ID NO: 1.

According to some embodiments, each monomer of the multimeric peptide comprises at least 19 consecutive amino acids from the sequence as set forth in SEQ ID NO: 1.

According to some embodiments, each monomer of the multimeric peptide comprises at least 20 consecutive amino acids from the sequence as set forth in SEQ ID NO: 1.

According to some embodiments, each monomer of the multimeric peptide comprises at least 21 consecutive amino acids from the sequence as set forth in SEQ ID NO: 1.

According to some embodiments, each monomer of the multimeric peptide comprises at least 22 consecutive amino acids from the sequence as set forth in SEQ ID NO: 1.

According to some embodiments, each monomer of the multimeric peptide comprises at least 23 consecutive amino acids from the sequence as set forth in SEQ ID NO: 1.

According to some embodiments, each monomer of the multimeric peptide comprises the full length sequence as set forth in SEQ ID NO: 1.

According to a particular embodiment the amino acid sequence derived from SEQ ID NO: 1 is HSIPTPILIFRSP (SEQ ID NO: 2), HLLRPRRRKRPHSI (SEQ ID NO: 3), RPRRRKRPHSIP (SEQ ID NO: 4), SIPTPILIFRSP (SEQ ID NO: 5), PHSIPTPILIFRSP (SEQ ID NO: 6) or HHLLRPRRRKR (SEQ ID NO: 7).

Preferably, each monomer of the multimeric peptide comprises at least 6, at least 7, at least 8, at least 9, at least 10, at least 11, at least 12, at least 13, at least 14 consecutive amino acids from the sequence as set forth in SEQ ID NO: 14—RPHSIPTPILIFRSP.

Additional contemplated peptides include those set forth in Table 1, herein below.

TABLE 1

| SEQ ID | Sequence |
|---|---|
| 15 | His-His-Leu-Leu-Arg-Pro |
| 16 | His-Leu-Leu-Arg-Pro-Arg |
| 17 | Leu-Leu-Arg-Pro-Arg-Arg |
| 18 | Leu-Arg-Pro-Arg-Arg-Lys |
| 19 | Arg-Pro-Arg-Arg-Lys-Arg |
| 20 | Pro-Arg-Arg-Lys-Arg-Pro |
| 21 | Arg-Arg-Lys-Arg-Pro-His |
| 22 | Arg-Lys-Arg-Pro-His-Ser |
| 23 | Lys-Arg-Pro-His-Ser-Ile |
| 24 | Arg-Pro-His-Ser-Ile-Pro |
| 25 | Pro-His-Ser-Ile-Pro-Thr |
| 26 | His-Ser-Ile-Pro-Thr-Pro |
| 27 | Ser-Ile-Pro-Thr-Pro-Ile |
| 28 | Ile-Pro-Thr-Pro-Ile-Leu |
| 29 | Pro-Thr-Pro-Ile-Leu-Ile |
| 30 | Thr-Pro-Ile-Leu-Ile-Phe |
| 31 | Pro-Ile-Leu-Ile-Phe-Arg |
| 32 | Ile-Leu-Ile-Phe-Arg-Ser |
| 33 | Leu-Ile-Phe-Arg-Ser-Pro |
| 34 | His-His-Leu-Leu-Arg-Pro-Arg |
| 35 | His-Leu-Leu-Arg-Pro-Arg-Arg |
| 36 | Leu-Leu-Arg-Pro-Arg-Arg-Lys |
| 37 | Leu-Arg-Pro-Arg-Arg-Lys-Arg |
| 38 | Arg-Pro-Arg-Arg-Lys-Arg-Pro |
| 39 | Pro-Arg-Arg-Lys-Arg-Pro-His |
| 40 | Arg-Arg-Lys-Arg-Pro-His-Ser |
| 41 | Arg-Lys-Arg-Pro-His-Ser-Ile |
| 42 | Lys-Arg-Pro-His-Ser-Ile-Pro |
| 43 | Arg-Pro-His-Ser-Ile-Pro-Thr |
| 44 | Pro-His-Ser-Ile-Pro-Thr-Pro |
| 45 | His-Ser-Ile-Pro-Thr-Pro-Ile |
| 46 | Ser-Ile-Pro-Thr-Pro-Ile-Leu |
| 47 | Ile-Pro-Thr-Pro-Ile-Leu-Ile |
| 48 | Pro-Thr-Pro-Ile-Leu-Ile-Phe |
| 49 | Thr-Pro-Ile-Leu-Ile-Phe-Arg |
| 50 | Pro-Ile-Leu-Ile-Phe-Arg-Ser |
| 51 | Ile-Leu-Ile-Phe-Arg-Ser-Pro |
| 52 | His-His-Leu-Leu-Arg-Pro-Arg-Arg |
| 53 | His-Leu-Leu-Arg-Pro-Arg-Arg-Lys |
| 54 | Leu-Leu-Arg-Pro-Arg-Arg-Lys-Arg |
| 55 | Leu-Arg-Pro-Arg-Arg-Lys-Arg-Pro |
| 56 | Arg-Pro-Arg-Arg-Lys-Arg-Pro-His |
| 57 | Pro-Arg-Arg-Lys-Arg-Pro-His-Ser |
| 58 | Arg-Arg-Lys-Arg-Pro-His-Ser-Ile |
| 59 | Arg-Lys-Arg-Pro-His-Ser-Ile-Pro |
| 60 | Lys-Arg-Pro-His-Ser-Ile-Pro-Thr |
| 61 | Arg-Pro-His-Ser-Ile-Pro-Thr-Pro |
| 62 | Pro-His-Ser-Ile-Pro-Thr-Pro-Ile |
| 63 | His-Ser-Ile-Pro-Thr-Pro-Ile-Leu |
| 64 | Ser-Ile-Pro-Thr-Pro-Ile-Leu-Ile |
| 65 | Ile-Pro-Thr-Pro-Ile-Leu-Ile-Phe |
| 66 | Pro-Thr-Pro-Ile-Leu-Ile-Phe-Arg |
| 67 | Thr-Pro-Ile-Leu-Ile-Phe-Arg-Ser |
| 68 | Pro-Ile-Leu-Ile-Phe-Arg-Ser-Pro |
| 69 | His-His-Leu-Leu-Arg-Pro-Arg-Arg-Lys |
| 70 | His-Leu-Leu-Arg-Pro-Arg-Arg-Lys-Arg |
| 71 | Leu-Leu-Arg-Pro-Arg-Arg-Lys-Arg-Pro |
| 72 | Leu-Arg-Pro-Arg-Arg-Lys-Arg-Pro-His |
| 73 | Arg-Pro-Arg-Arg-Lys-Arg-Pro-His-Ser |
| 74 | Pro-Arg-Arg-Lys-Arg-Pro-His-Ser-Ile |
| 75 | Arg-Arg-Lys-Arg-Pro-His-Ser-Ile-Pro |
| 76 | Arg-Lys-Arg-Pro-His-Ser-Ile-Pro-Thr |
| 77 | Lys-Arg-Pro-His-Ser-Ile-Pro-Thr-Pro |
| 78 | Arg-Pro-His-Ser-Ile-Pro-Thr-Pro-Ile |
| 79 | Pro-His-Ser-Ile-Pro-Thr-Pro-Ile-Leu |
| 80 | His-Ser-Ile-Pro-Thr-Pro-Ile-Leu-Ile |
| 81 | Ser-Ile-Pro-Thr-Pro-Ile-Leu-Ile-Phe |
| 82 | Ile-Pro-Thr-Pro-Ile-Leu-Ile-Phe-Arg |
| 83 | Pro-Thr-Pro-Ile-Leu-Ile-Phe-Arg-Ser |
| 84 | Thr-Pro-Ile-Leu-Ile-Phe-Arg-Ser-Pro |
| 85 | His-His-Leu-Leu-Arg-Pro-Arg-Arg-Lys-Arg |
| 86 | His-Leu-Leu-Arg-Pro-Arg-Arg-Lys-Arg-Pro |
| 87 | Leu-Leu-Arg-Pro-Arg-Arg-Lys-Arg-Pro-His |
| 88 | Leu-Arg-Pro-Arg-Arg-Lys-Arg-Pro-His-Ser |
| 89 | Arg-Pro-Arg-Arg-Lys-Arg-Pro-His-Ser-Ile |
| 90 | Pro-Arg-Arg-Lys-Arg-Pro-His-Ser-Ile-Pro |
| 91 | Arg-Arg-Lys-Arg-Pro-His-Ser-Ile-Pro-Thr |
| 92 | Arg-Lys-Arg-Pro-His-Ser-Ile-Pro-Thr-Pro |
| 93 | Lys-Arg-Pro-His-Ser-Ile-Pro-Thr-Pro-Ile |
| 94 | Arg-Pro-His-Ser-Ile-Pro-Thr-Pro-Ile-Leu |
| 95 | Pro-His-Ser-Ile-Pro-Thr-Pro-Ile-Leu-Ile |
| 96 | His-Ser-Ile-Pro-Thr-Pro-Ile-Leu-Ile-Phe |
| 97 | Ser-Ile-Pro-Thr-Pro-Ile-Leu-Ile-Phe-Arg |
| 98 | Ile-Pro-Thr-Pro-Ile-Leu-Ile-Phe-Arg-Ser |
| 99 | Pro-Thr-Pro-Ile-Leu-Ile-Phe-Arg-Ser-Pro |

According to a particular embodiment, each peptide monomer has the sequence as disclosed in SEQ ID NO: 101.

The term “peptide” as used herein refers to a polymer of natural or synthetic amino acids, encompassing native peptides (either degradation products, synthetically synthesized peptides or recombinant peptides) and peptidomimetics (typically, synthetically synthesized peptides), as well as peptoids and semipeptoids which are peptide analogs, which may have, for example, modifications rendering the peptides even more stable while in a body or more capable of penetrating into cells.

Such modifications include, but are not limited to N terminus modification, C terminus modification, peptide bond modification, including, but not limited to, CH2-NH, CH2-S, CH2-S═O, O═C—NH, CH2-O, CH2-CH2, S═C—NH, CH═CH or CF═CH, backbone modifications, and residue modification. Methods for preparing peptidomimetic compounds are well known in the art and are specified, for example, in Quantitative Drug Design, C. A. Ramsden Gd., Chapter 17.2, F. Choplin Pergamon Press (1992), which is incorporated by reference as if fully set forth herein. Further details in this respect are provided hereinunder.

Peptide bonds (—CO—NH—) within the peptide may be substituted, for example, by N-methylated bonds (—N(CH3)-CO—), ester bonds (—C(R)H—C—O—O—C(R)—N—), ketomethylen bonds (—CO—CH2-), α-aza bonds (—NH—N(R)—CO—), wherein R is any alkyl, e.g., methyl, carba bonds (—CH2-NH—), hydroxyethylene bonds (—CH(OH)—CH2), thioamide bonds (—CS—NH—), olefinic double bonds (—CH═CH—), retro amide bonds (—NH—CO—), peptide derivatives (—N(R)—CH2-CO—), wherein R is the “normal” side chain, naturally presented on the carbon atom.

These modifications can occur at any of the bonds along the peptide chain and even at several (2-3) at the same time.

Natural aromatic amino acids, Trp, Tyr and Phe, may be substituted for synthetic non-natural acid such as Phenylglycine, TIC, naphthylelanine (Nol), ring-methylated derivatives of Phe, halogenated derivatives of Phe or o-methyl-Tyr.

In addition to the above, the peptides of the present invention may also include one or more modified amino acids or one or more non-amino acid monomers (e.g. fatty acids, complex carbohydrates etc.).

As used herein in the specification and in the claims section below the term “amino acid” or “amino acids” is understood to include the 20 naturally occurring amino acids; those amino acids often modified post-translationally in vivo, including, for example, hydroxyproline, phosphoserine and phosphothreonine; and other unusual amino acids including, but not limited to, 2-aminoadipic acid, hydroxylysine, isodemosine, nor-valine, nor-leucine and ornithine. Furthermore, the term “amino acid” includes both D- and L-amino acids (stereoisomers).

Tables 2 and 3 below list naturally occurring amino acids (Table 2) and non-conventional or modified amino acids (Table 3) which can be used with the present invention.

TABLE 2

| One-letter | Three-Letter | |
|---|---|---|
| Symbol | Abbreviation | Amino Acid |
| A | Ala | alanine |
| R | Arg | Arginine |
| N | Asn | Asparagine |
| D | Asp | Aspartic acid |
| C | Cys | Cysteine |
| Q | Gln | Glutamine |
| E | Glu | Glutamic Acid |
| G | Gly | glycine |
| H | His | Histidine |
| I | Iie | isoleucine |
| L | Leu | leucine |
| K | Lys | Lysine |
| M | Met | Methionine |
| F | Phe | phenylalanine |
| P | Pro | Proline |
| S | Ser | Serine |
| T | Thr | Threonine |
| W | Trp | tryptophan |
| Y | Tyr | tyrosine |
| V | Val | Valine |
| X | Xaa | Any amino acid as above |

TABLE 3

| Code | Non-conventional amino acid | Code | Non-conventional amino acid |
|---|---|---|---|
| Nmala | L-N-methylalanine | Abu | α-aminobutyric acid |
| Nmarg | L-N-methylarginine | Mgabu | α-amino-α-methylbutyrate |
| Nmasn | L-N-methylasparagine | Cpro | aminocyclopropane- |
| Nmasp | L-N-methylaspartic acid | | carboxylate |
| Nmcys | L-N-methylcysteine | Aib | aminoisobutyric acid |
| Nmgin | L-N-methylglutamine | Norb | aminonorbornyl- |
| Nmglu | L-N-methylglutamic acid | | carboxylate |
| Nmhis | L-N-methylhistidine | Chexa | cyclohexylalanine |
| Nmile | L-N-methylisolleucine | Cpen | cyclopentylalanine |
| Nmleu | L-N-methylleucine | Dal | D-alanine |
| Nmlys | L-N-methyllysine | Darg | D-arginine |
| Nmmet | L-N-methylmethionine | Dasp | D-aspartic acid |
| Nmnle | L-N-methylnorleucine | Dcys | D-cysteine |
| Nmnva | L-N-methylnorvaline | Dgln | D-glutamine |
| Nmorn | L-N-methylornithine | Dglu | D-glutamic acid |
| Nmphe | L-N-methylphenylalanine | Dhis | D-histidine |
| Nmpro | L-N-methylproline | Dile | D-isoleucine |
| Nmser | L-N-methylserine | Dleu | D-leucine |
| Nmthr | L-N-methylthreonine | Dlys | D-lysine |
| Nmtrp | L-N-methyltryptophan | Dmet | D-methionine |
| Nmtyr | L-N-methyltyrosine | Dorn | D-ornithine |
| Nmval | L-N-methylvaline | Dphe | D-phenylalanine |
| Nmetg | L-N-methylethylglycine | Dpro | D-proline |
| Nmtbug | L-N-methyl-t-butylglycine | Dser | D-serine |
| Nle | L-norleucine | Dthr | D-threonine |
| Nva | L-norvaline | Dtrp | D-tryptophan |
| Maib | α-methyl-aminoisobutyrate | Dtyr | D-tyrosine |
| Mgabu | α-methyl-γ-aminobutyrate | Dval | D-valine |
| Mchexa | α ethylcyclohexylalanine | Dmala | D-α-methylalanine |
| Mcpen | α-methylcyclopentylalanine | Dmarg | D-α-methylarginine |
| Manap | α-methyl-α-napthylalanine | Dmasn | D-α-methylasparagine |
| Mpen | α-methylpenicillamine | Dmasp | D-α-methylaspartate |
| Nglu | N-(4-aminobutyl)glycine | Dmcys | D-α-methylcysteine |
| Naeg | N-(2-aminoethyl)glycine | Dmgln | D-α-methylglutamine |
| Norn | N-(3-aminopropyl)glycine | Dmhis | D-α-methylhistidine |
| Nmaabu | N-amino-α-methylbutyrate | Dmile | D-α-methylisoleucine |
| Anap | α-napthylalanine | Dmleu | D-α-methylleucine |
| Nphe | N-benzylglycine | Dmlys | D-α-methyllysine |
| Ngln | N-(2-carbamylethyl)glycine | Dmmet | D-α-methylmethionine |
| Nasn | N-(carbamylmethyl)glycine | Dmorn | D-α-methylornithine |
| Nglu | N-(2-carboxyethyl)glycine | Dmphe | D-α-methylphenylalanine |
| Nasp | N-(carboxymethyl)glycine | Dmpro | D-α-methylproline |
| Ncbut | N-cyclobutylglycine | Dmser | D-α-methylserine |
| Nchep | N-cycloheptylglycine | Dmthr | D-α-methylthreonine |
| Nchex | N-cyclohexylglycine | Dmtrp | D-α-methyltryptophan |
| Ncdec | N-cyclodecylglycine | Dmty | D-α-methyltyrosine |
| Ncdod | N-cyclododeclglycine | Dmval | D-α-methylvaline |
| Ncoct | N-cyclooctylglycine | Dnmala | D-α-methylalnine |
| Ncpro | N-cyclopropylglycine | Dnmarg | D-α-methylarginine |
| Ncund | N-cycloundecylglycine | Dnmasn | D-α-methylasparagine |
| Nbhm | N-(2,2-diphenylethyl)glycine | Dnmasp | D-α-methylasparatate |
| Nbhe | N-(3,3- | Dnmcys | D-α-methylcysteine |
| | diphenylpropyl)glycine | | |
| Nhtrp | N-(3-indolylyethyl) glycine | Dnmleu | D-N-methylleucine |
| Nmgabu | N-methyl-γ-aminobutyrate | Dnmlys | D-N-methyllysine |
| Dnmmet | D-N-methylmethionine | Nmchexa | N-methylcyclohexylalanine |
| Nmcpen | N-methylcyclopentylalanine | Dnmorn | D-N-methylornithine |
| Dnmphe | D-N-methylphenylalanine | Nala | N-methylglycine |
| Dnmpro | D-N-methylproline | Nmaib | N-methylaminoisobutyrate |
| Dnmser | D-N-methylserine | Nile | N-(1-methylpropyl)glycine |
| Dnmser | D-N-methylserine | Nile | N-(2-methylpropyl)glycine |
| Dnmthr | D-N-methylthreonine | Nleu | N-(2-methylpropyl)glycine |
| Nva | N-(1-methylethyl)glycine | Dnmtrp | D-N-methyltryptophan |
| Nmanap | N-methyla-napthylalanine | Dnmtyr | D-N-methyltyrosine |
| Nmpen | N-methylpenicillamine | Dnmval | D-N-methylvaline |
| Nhtyr | N-(p-hydroxyphenyl)glycine | Gabu | γ-aminobutyric acid |
| Ncys | N-(thiomethyl)glycine | Tbug | L-t-butylglycine |
| Pen | penicillamine | Etg | L-ethylglycine |
| Mala | L-α-methylalanine | Hphe | L-homophenylalanine |
| Masn | L-α-methylasparagine | Marg | L-α-methylarginine |
| Mtbug | L-α-methyl-t-butylglycine | Masp | L-α-methylaspartate |
| Metg | L-methylethylglycine | Mcys | L-α-methylcysteine |
| Mglu | L-α-methylglutamate | Mgln | L-α thylglutamine |
| Mhphe | L-α-methylhomophenylalanine | Mhis | L-α-methylhistidine |
| Nmet | N-(2-methylthioethyl)glycine | Mile | L-α-methylisoleucine |
| Narg | N-(3-guanidinopropyl)glycine | Dnmgln | D-N-methylglutamine |
| Nthr | N-(1-hydroxyethyl)glycine | Dnmglu | D-N-methylglutamate |
| Nser | N-(hydroxyethyl)glycine | Dnmhis | D-N-methylhistidine |
| Nhis | N-(imidazolylethyl)glycine | Dnmile | D-N-methylisoleucine |
| Nhtrp | N-(3-indolylyethyl)glycine | Dnmleu | D-N-methylleucine |
| Nmgabu | N-methyl-γ-aminobutyrate | Dnmlys | D-N-methyllysine |
| Dnmmet | D-N-methylmethionine | Nmchexa | N-methylcyclohexylalanine |
| Nmcpen | N-methylcyclopentylalanine | Dnmorn | D-N-methylornithine |
| Dnmphe | D-N-methylphenylalanine | Nala | N-methylglycine |
| Dnmpro | D-N-methylproline | Nmaib | N-methylaminoisobutyrate |
| Dnmser | D-N-methylserine | Nile | N-(1-methylpropyl)glycine |
| Dnmthr | D-N-methylthreonine | Nleu | N-(2-methylpropyl)glycine |
| Nval | N-(1-methylethyl)glycine | Dnmtrp | D-N-methyltryptophan |
| Nmanap | N-methyla-napthylalanine | Dnmtyr | D-N-methyltyrosine |
| Nmpen | N-methylpenicillamine | Dnmval | D-N-methylvaline |
| Nhtyr | N-(p-hydroxyphenyl)glycine | Gabu | γ-aminobutyric acid |
| Ncys | N-(thiomethyl)glycine | Tbug | L-t-butylglycine |
| Pen | penicillamine | Etg | L-ethylglycine |
| Mala | L-α-methylalanine | Hphe | L-homophenylalanine |
| Masn | L-α-methylasparagine | Marg | L-α-methylarginine |
| Mtbug | L-α-methyl-t-butylglycine | Masp | L-α-methylaspartate |
| Metg | L-methylethylglycine | Mcys | L-α-methylcysteine |
| Mglu | L-α-methylglutamate | Mgln | L-α-methylglutamine |
| Mhphe | L-α-methylhomophenylalanine | Mhis | L-α ethylhistidine |
| Nmet | N-(2-methylthioethyl)glycine | Mile | L-α thylisoleucine |
| Mlys | L-α-methyllysine | Mleu | L-α-methylleucine |
| Mnle | L-α-methylnorleucine | Mmet | L-α-methylmethionine |
| Morn | L-α-methylornithine | Mnva | L-α-methylnorvaline |
| Mpro | L-α-methylproline | Mphe | L-α-methylphenylalanine |
| Mthr | L-α-methylthreonine | mser | L-α-methylserine |
| Mtyr | L-α-methyltyrosine | Mtrp | L-α ethylvaline |
| Nmhphe | L-N-methylhomophenylalanine | Mval | L-α-methylleucine |
| | N-(N-(3,3-diphenylpropyl) | Nnbhm | N-(N-(2,2-diphenylethyl) |
| Nnbhe | carbamylmethyl(1)glycine | Nnbhm | carbamylmethyl-glycine |
| | | Nmbc | 1-carboxy-1-(2,2-diphenyl |
| | | | ethylamino)cyclopropane |

It will be appreciated that additional peptides are contemplated by the present invention as well as those disclosed herein, which may be synthesized (comprising conservative or non-conservative substitutions) in order to “tweak” the peptides and generate peptides with improved characteristics e.g., comprising an enhanced ability to bind to CD45 and/or to stimulate the secretion of IFN gamma from T lymphocytes.

Thus, in other embodiments, the peptide monomers comprise a homolog, a variant, or a functional fragment of the sequences described herein above. In another embodiment, the peptide monomers comprise an amino acid sequence that is about 60%, 65%, 70%, 75%, 80%, 85%, 90%, 95%, 96%, 97%, 98%, 99% identical to the sequences described herein above.

The term “conservative substitution” as used herein, refers to the replacement of an amino acid present in the native sequence in the peptide with a naturally or non-naturally occurring amino or a peptidomimetics having similar steric properties. Where the side-chain of the native amino acid to be replaced is either polar or hydrophobic, the conservative substitution should be with a naturally occurring amino acid, a non-naturally occurring amino acid or with a peptidomimetic moiety which is also polar or hydrophobic (in addition to having the same steric properties as the side-chain of the replaced amino acid).

As naturally occurring amino acids are typically grouped according to their properties, conservative substitutions by naturally occurring amino acids can be easily determined bearing in mind the fact that in accordance with the invention replacement of charged amino acids by sterically similar non-charged amino acids are considered as conservative substitutions.

For producing conservative substitutions by non-naturally occurring amino acids it is also possible to use amino acid analogs (synthetic amino acids) well known in the art. A peptidomimetic of the naturally occurring amino acid is well documented in the literature known to the skilled practitioner.

When affecting conservative substitutions the substituting amino acid should have the same or a similar functional group in the side chain as the original amino acid.

The phrase “non-conservative substitutions” as used herein refers to replacement of the amino acid as present in the parent sequence by another naturally or non-naturally occurring amino acid, having different electrochemical and/or steric properties. Thus, the side chain of the substituting amino acid can be significantly larger (or smaller) than the side chain of the native amino acid being substituted and/or can have functional groups with significantly different electronic properties than the amino acid being substituted. Examples of non-conservative substitutions of this type include the substitution of phenylalanine or cyclohexylmethyl glycine for alanine, isoleucine for glycine, or —NH—CH[(—CH₂)₅—COOH]—CO— for aspartic acid. Those non-conservative substitutions which fall under the scope of the present invention are those which still constitute a peptide having anti-bacterial properties.

The N and C termini of the peptides of the present invention may be protected by function groups. Suitable functional groups are described in Green and Wuts, “Protecting Groups in Organic Synthesis”, John Wiley and Sons, Chapters 5 and 7, 1991, the teachings of which are incorporated herein by reference.

Hydroxyl protecting groups include esters, carbonates and carbamate protecting groups. Amine protecting groups include alkoxy and aryloxy carbonyl groups, as described above for N-terminal protecting groups. Carboxylic acid protecting groups include aliphatic, benzylic and aryl esters, as described above for C-terminal protecting groups. In one embodiment, the carboxylic acid group in the side chain of one or more glutamic acid or aspartic acid residue in a peptide of the present invention is protected, preferably with a methyl, ethyl, benzyl or substituted benzyl ester.

Examples of N-terminal protecting groups include acyl groups (—CO—R1) and alkoxy carbonyl or aryloxy carbonyl groups (—CO—O—R1), wherein R1 is an aliphatic, substituted aliphatic, benzyl, substituted benzyl, aromatic or a substituted aromatic group. Specific examples of acyl groups include acetyl, (ethyl)-CO—, n-propyl-CO—, iso-propyl-CO—, n-butyl-CO—, sec-butyl-CO—, t-butyl-CO—, hexyl, lauroyl, palmitoyl, myristoyl, stearyl, oleoyl phenyl-CO—, substituted phenyl-CO—, benzyl-CO— and (substituted benzyl)-CO—. Examples of alkoxy carbonyl and aryloxy carbonyl groups include CH3-O—CO—, (ethyl)-O—CO—, n-propyl-O—CO—, iso-propyl-O—CO—, n-butyl-O—CO—, sec-butyl-O—CO—, t-butyl-O—CO—, phenyl-O— CO—, substituted phenyl-O—CO— and benzyl-O—CO—, (substituted benzyl)-O—CO—. Adamantan, naphtalen, myristoleyl, tuluen, biphenyl, cinnamoyl, nitrobenzoy, toluoyl, furoyl, benzoyl, cyclohexane, norbornane, Z-caproic. In order to facilitate the N-acylation, one to four glycine residues can be present in the N-terminus of the molecule.

The carboxyl group at the C-terminus of the compound can be protected, for example, by an amide (i.e., the hydroxyl group at the C-terminus is replaced with —NH₂, —NHR₂ and —NR₂R₃) or ester (i.e. the hydroxyl group at the C-terminus is replaced with —OR₂). R₂ and R₃ are independently an aliphatic, substituted aliphatic, benzyl, substituted benzyl, aryl or a substituted aryl group. In addition, taken together with the nitrogen atom, R₂ and R₃ can form a C4 to C8 heterocyclic ring with from about 0-2 additional heteroatoms such as nitrogen, oxygen or sulfur. Examples of suitable heterocyclic rings include piperidinyl, pyrrolidinyl, morpholino, thiomorpholino or piperazinyl. Examples of C-terminal protecting groups include —NH₂, —NHCH₃, —N(CH₃)₂, —NH(ethyl), —N(ethyl)₂, —N(methyl) (ethyl), —NH(benzyl), —N(C1-C4 alkyl)(benzyl), —NH(phenyl), —N(C1-C4 alkyl) (phenyl), —OCH₃, —O-(ethyl), —O-(n-propyl), —O-(n-butyl), —O-(iso-propyl), —O-(sec-butyl), —O-(t-butyl), —O-benzyl and —O-phenyl.

The peptides of the present invention may also comprise non-amino acid moieties, such as for example, hydrophobic moieties (various linear, branched, cyclic, polycyclic or hetrocyclic hydrocarbons and hydrocarbon derivatives) attached to the peptides; various protecting groups, especially where the compound is linear, which are attached to the compound's terminals to decrease degradation. Chemical (non-amino acid) groups present in the compound may be included in order to improve various physiological properties such; decreased degradation or clearance; decreased repulsion by various cellular pumps, improve immunogenic activities, improve various modes of administration (such as attachment of various sequences which allow penetration through various barriers, through the gut, etc.); increased specificity, increased affinity, decreased toxicity and the like.

Exemplary side chain protecting groups and their positioning are described in the Examples section herein below.

Linking of the monomers of the peptide may be effected using any method known in the art provided that the linking does not substantially interfere with the bioactivity of the multimeric peptide—e.g. to interfere with the ability of the multimeric peptide to bind to CD45 or enhance secretion of interferon gamma (INF-γ) from activated leukocytes.

The monomers of this aspect of the present invention may be linked through a linking moiety.

Examples of linking moieties include but are not limited to a simple covalent bond, a flexible peptide linker, a disulfide bridge or a polymer such as polyethylene glycol (PEG). Peptide linkers may be entirely artificial (e.g., comprising 2 to 20 amino acid residues independently selected from the group consisting of glycine, serine, asparagine, threonine and alanine) or adopted from naturally occurring proteins. Disulfide bridge formation can be achieved, e.g., by addition of cysteine residues, as further described herein below. Linking through polyethylene glycols (PEG) can be achieved by reaction of monomers having free cysteines with multifunctional PEGs, such as linear bis-maleimide PEGs. Alternatively, linking can be performed though the glycans on the monomer after their oxidation to aldehyde form and using multifunctional PEGs containing aldehyde-reactive groups.

Selection of the position of the link between the two monomers should take into account that the link should not substantially interfere with the ability of the multimer to enhance the secretion of interferon gamma (INF-γ) from activated leukocytes and/or to bind to CD45.

Thus, for example, the linking moiety is optionally a moiety which is covalently attached to a side chain, an N-terminus or a C-terminus of the first peptide monomer, as well as to a side chain, an N-terminus or a C-terminus of the second peptide monomer.

Preferably the linking moiety is attached to the C-terminus of the first peptide monomer, and to the C-terminus of the second peptide monomer.

As mentioned, the linking moiety used in this aspect of the present invention may be a cysteine residue.

Thus, in some embodiments of the invention, each of the peptide monomers comprises an amino acid sequence as described herein above and further comprise at least one cysteine residue, such that the peptide monomers are covalently linked to one another via a disulfide bridge formed between a cysteine residue in one peptide monomer and a cysteine residue in another peptide monomer.

Typically, the cysteine is situated at the carboxy end of the peptide monomers.

Hereinthroughout, the phrases “disulfide bridge” and “disulfide bond” are used interchangeably, and describe a —S—S— bond.

The linker may comprise additional amino acids linked together by peptide bonds which serve as spacers such that the linker does not interfere with the biological activity of the final compound. The linker is preferably made up of amino acids linked together by peptide bonds. Thus, in preferred embodiments, the linker is made up of from 1 to 10 amino acids linked by peptide bonds, wherein the amino acids are selected from the 20 naturally occurring amino acids. Some of these amino acids may be glycosylated, as is well understood by those in the art. In a more preferred embodiment, besides cysteine the amino acids in the linker are selected from glycine, alanine, proline, asparagine, glutamine, and lysine. Even more preferably, besides cysteine, the linker is made up of a majority of amino acids that are sterically unhindered, such as glycine and alanine.

Thus, according to one embodiment the linker comprises the sequence cysteine-glycine.

Exemplary monomer sequences are thus set forth by the following sequences:

(SEQ ID NO: 8)
CGHSIPTPILIFRSP,
(SEQ ID NO: 9)
CGHLLRPRRRKRPHSI,
(SEQ ID NO: 10)
CGRPRRRKRPHSIP,
(SEQ ID NO: 11)
CGSIPTPILIFRSP,
(SEQ ID NO: 12)
CGPHSIPTPILIFRSP
or
(SEQ ID NO: 13)
CGHHLLRPRRRKR.

Non-peptide linkers are also possible. For example, alkyl linkers such as —NH—(CH.sub.2).sub.s--C(O)—, wherein s=2-20 could be used. These alkyl linkers may further be substituted by any non-sterically hindering group such as lower alkyl (e.g., C₁-C₆) lower acyl, halogen (e.g., Cl, Br), CN, NH₂, phenyl, etc. An exemplary non-peptide linker is a PEG linker.

Thus, in some embodiments, at least one of monomers is PEGylated or chemically modified to another form. PEGylation of the molecules can be carried out, e.g., according to the methods described in Youngster et al., Curr Pharm Des (2002), 8:2139; Grace et al., J Interferon Cytokine Res (2001), 21: 1103; Pepinsky et al., J Pharmacol Exp Ther (2001), 297:1059; Pettit et al., J Biol Chem (1997), 272:2312; Goodson et al. Biotechnology NY (1990), 8:343; Katre; J Immunol (1990), 144:209, Behrens et al US2006/0198819 A1, Klausen et al US2005/0113565 A1.

Any kind of polyethylene glycol is suitable for the present invention provided that the PEG-polypeptide-oligomer is still capable of binding to CD45 which can be assayed according to methods known in the art.

Preferably, the polyethylene glycol of the polypeptide-dimer of the present invention is PEG 1000, 2000, 3000, 5000, 10000, 15000, 20000 or 40000 with PEG 20000 or 40000 being particularly preferred.

According to another embodiment the link is effected using a coupling agent.

The term “coupling agent”, as used herein, refers to a reagent that can catalyze or form a bond between two or more functional groups intra-molecularly, inter-molecularly or both. Coupling agents are widely used to increase polymeric networks and promote crosslinking between polymeric chains, hence, in the context of some embodiments of the present invention, the coupling agent is such that can promote crosslinking between polymeric chains; or such that can promote crosslinking between amino functional groups and carboxylic functional groups, or between other chemically compatible functional groups of polymeric chains. In some embodiments of the present invention the term “coupling agent” may be replaced with the term “crosslinking agent”. In some embodiments, one of the polymers serves as the coupling agent and acts as a crosslinking polymer.

By “chemically compatible” it is meant that two or more types of functional groups can react with one another so as to form a bond.

Exemplary functional groups which are typically present in gelatins and alginates include, but are not limited to, amines (mostly primary amines —NH₂), carboxyls (—CO₂H), sulfhydryls and hydroxyls (—SH and —OH respectively), and carbonyls (—COH aldehydes and —CO— ketones).

Primary amines occur at the N-terminus of polypeptide chains (called the alpha-amine), at the side chain of lysine (Lys, K) residues (the epsilon-amine), as found in gelatin, as well as in various naturally occurring polysaccharides and aminoglycosides. Because of its positive charge at physiologic conditions, primary amines are usually outward-facing (i.e., found on the outer surface) of proteins and other macromolecules; thus, they are usually accessible for conjugation.

Carboxyls occur at the C-terminus of polypeptide chain, at the side chains of aspartic acid (Asp, D) and glutamic acid (Glu, E), as well as in naturally occurring aminoglycosides and polysaccharides such as alginate. Like primary amines, carboxyls are usually on the surface of large polymeric compounds such as proteins and polysaccharides.

Sulfhydryls and hydroxyls occur in the side chain of cysteine (Cys, C) and serine, (Ser, S) respectively. Hydroxyls are abundant in polysaccharides and aminoglycosides.

Carbonyls as ketones or aldehydes can be form in glycoproteins, glycosides and polysaccharides by various oxidizing processes, synthetic and/or natural.

According to some embodiments of the present invention, the coupling agent can be selected according to the type of functional groups and the nature of the crosslinking bond that can be formed therebetween. For example, carboxyl coupling directly to an amine can be afforded using a carbodiimide type coupling agent, such as EDC; amines may be coupled to carboxyls, carbonyls and other reactive functional groups by N-hydroxysuccinimide esters (NHS-esters), imidoester, PFP-ester or hydroxymethyl phosphine; sulfhydryls may be coupled to carboxyls, carbonyls, amines and other reactive functional groups by maleimide, haloacetyl (bromo- or iodo-), pyridyldisulfide and vinyl sulfone; aldehydes as in oxidized carbohydrates, may be coupled to other reactive functional groups with hydrazide; and hydroxyl may be coupled to carboxyls, carbonyls, amines and other reactive functional groups with isocyanate.

Hence, suitable coupling agents that can be used in some embodiments of the present invention include, but are not limited to, carbodiimides, NHS-esters, imidoesters, PFP-esters or hydroxymethyl phosphines.

The peptides of the present invention can be biochemically synthesized such as by using standard solid phase techniques. These methods include exclusive solid phase synthesis, partial solid phase synthesis methods, fragment condensation, classical solution synthesis. Solid phase polypeptide synthesis procedures are well known in the art and further described by John Morrow Stewart and Janis Dillaha Young, Solid Phase Polypeptide Syntheses (2nd Ed., Pierce Chemical Company, 1984).

Synthetic peptides can be purified by preparative high performance liquid chromatography [Creighton T. (1983) Proteins, structures and molecular principles. WH Freeman and Co. N.Y.] and the composition of which can be confirmed via amino acid sequencing.

Recombinant techniques may also be used to generate the monomers of the present invention. To produce a peptide of the present invention using recombinant technology, a polynucleotide encoding the monomer of the present invention is ligated into a nucleic acid expression vector, which comprises the polynucleotide sequence under the transcriptional control of a cis-regulatory sequence (e.g., promoter sequence) suitable for directing constitutive, tissue specific or inducible transcription of the monomers of the present invention in the host cells.

In addition to being synthesizable in host cells, the monomers of the present invention can also be synthesized using in vitro expression systems. These methods are well known in the art and the components of the system are commercially available.

Typically, the monomers are synthesized as individual peptides, following which, depending on the linking moiety present in the monomers, linking is effected. For example, if the linking moiety is a cysteine residue, thiol oxidation is performed.

When Cys residue is used as a linking moiety, disulfide bonds may be formed by oxidation thereof. In one embodiment the control of cysteine bond formation is exercised by choosing an oxidizing agent of the type and concentration effective to optimize formation of the multimer. Examples of oxidizing agent include iodine, dimethylsulfoxide (DMSO), potassium ferricyanide, and the like.

If the monomers comprise two or more cysteine residues, isomers resulting from disulfide bonds of different binding manner may be erroneously obtained. A peptide dimer wherein a disulfide bond is formed between intended cysteine residues can be prepared by selecting a particular combination of protecting groups for cysteine side chains. Examples of the combination of protecting groups include MeBzl (methylbenzyl) and Acm (acetamidemethyl) groups, Trt (trityl) and Acm groups, Npys (3-nitro-2-pyridylthio) and Acm groups, S-Bu-t (S-tert-butyl) and Acm groups, and the like. For example, in the case of a combination of MeBzl and Acm groups, the preparation can be carried out by a method comprising removing protecting groups other than MeBzl group and a protecting group(s) on the cysteine side chain, and subjecting the resulting monomer solution to air-oxidation to form a disulfide bond(s) between the deprotected cysteine residues, followed by deprotection and oxidization with iodine to form a disulfide bond(s) between the cysteine residues previously protected by Acm.

In embodiments where a peptide dimer is dimerized via a linker moiety, the linker may be incorporated into the peptide during peptide synthesis. For example, where a linker moiety contains two functional groups capable of serving as initiation sites for peptide synthesis and a third functional group (e.g., a carboxyl group or an amino group) that enables binding to another molecular moiety, the linker may be conjugated to a solid support. Thereafter, two peptide monomers may be synthesized directly onto the two reactive nitrogen groups of the linker moiety in a variation of the solid phase synthesis technique.

In alternate embodiments where a peptide dimer is dimerized by a linker moiety, the linker may be conjugated to the two peptide monomers of a peptide dimer after peptide synthesis. Such conjugation may be achieved by methods well established in the art. In one embodiment, the linker contains at least two functional groups suitable for attachment to the target functional groups of the synthesized peptide monomers. For example, a linker with two free amine groups may be reacted with the C-terminal carboxyl groups of each of two peptide monomers. In another example, linkers containing two carboxyl groups, either preactivated or in the presence of a suitable coupling reagent, may be reacted with the N-terminal or side chain amine groups, or C-terminal lysine amides, of each of two peptide monomers.

Monomers of the invention can be attached to water-soluble polymers (e.g., PEG) using any of a variety of chemistries to link the water-soluble polymer(s) to the receptor-binding portion of the molecule (e.g., peptide+spacer). A typical embodiment employs a single attachment junction for covalent attachment of the water soluble polymer(s) to the receptor-binding portion, however in alternative embodiments multiple attachment junctions may be used, including further variations wherein different species of water-soluble polymer are attached to the receptor-binding portion at distinct attachment junctions, which may include covalent attachment junction(s) to the spacer and/or to one or both peptide chains. In some embodiments, the dimer or higher order multimer will comprise distinct species of peptide chain (i.e., a heterodimer or other heteromultimer). By way of example and not limitation, a dimer may comprise a first peptide chain having a PEG attachment junction and the second peptide chain may either lack a PEG attachment junction or utilize a different linkage chemistry than the first peptide chain and in some variations the spacer may contain or lack a PEG attachment junction and the spacer, if PEGylated, may utilize a linkage chemistry different than that of the first and/or second peptide chains. An alternative embodiment employs a PEG attached to the spacer portion of the receptor-binding portion and a different water-soluble polymer (e.g., a carbohydrate) conjugated to a side chain of one of the amino acids of the peptide portion of the molecule.

The peptides of the present invention may also comprise non-amino acid moieties, such as for example, hydrophobic moieties (various linear, branched, cyclic, polycyclic or heterocyclic hydrocarbons and hydrocarbon derivatives) attached to the peptides; various protecting groups, especially where the compound is linear, which are attached to the compound's terminals to decrease degradation. Chemical (non-amino acid) groups present in the compound may be included in order to improve various physiological properties such; decreased degradation or clearance; decreased repulsion by various cellular pumps, improve immunogenic activities, improve various modes of administration (such as attachment of various sequences which allow penetration through various barriers, through the gut, etc.); increased specificity, increased affinity, decreased toxicity and the like.

According to one embodiment, the peptides of the present invention are attached to a sustained-release enhancing agent. Exemplary sustained-release enhancing agents include, but are not limited to hyaluronic acid (HA), alginic acid (AA), polyhydroxyethyl methacrylate (Poly-HEMA), polyethylene glycol (PEG), glyme and polyisopropylacrylamide.

Attaching the amino acid sequence component of the peptides of the invention to other non-amino acid agents may be by covalent linking, by non-covalent complexion, for example, by complexion to a hydrophobic polymer, which can be degraded or cleaved producing a compound capable of sustained release; by entrapping the amino acid part of the peptide in liposomes or micelles to produce the final peptide of the invention. The association may be by the entrapment of the amino acid sequence within the other component (liposome, micelle) or the impregnation of the amino acid sequence within a polymer to produce the final peptide of the invention.

The peptides described herein may be used for treating subjects having diseases including autoimmune diseases, neurodegenerative diseases and infectious diseases. It will be appreciated that the autoimmune disease, neurodegenerative disease and infectious disease which can be treated are not cancerous diseases (except for triple negative breast cancer and head and neck cancer, which are specifically contemplated).

Autoimmune Diseases:

Examples of autoimmune diseases which can be treated by the polypeptides of the present invention include, but are not limited to cardiovascular diseases, rheumatoid diseases, glandular diseases, gastrointestinal diseases, cutaneous diseases, hepatic diseases, neurological diseases, muscular diseases, nephric diseases, diseases related to reproduction, connective tissue diseases and systemic diseases.

Examples of autoimmune cardiovascular diseases include, but are not limited to atherosclerosis (Matsuura E. et al., Lupus. 1998; 7 Suppl 2:S135), myocardial infarction (Vaarala O. Lupus. 1998; 7 Suppl 2:S132), thrombosis (Tincani A. et al., Lupus 1998; 7 Suppl 2:S107-9), Wegener's granulomatosis, Takayasu's arteritis, Kawasaki syndrome (Praprotnik S. et al., Wien Klin Wochenschr Aug. 25, 2000; 112 (15-16):660), anti-factor VIII autoimmune disease (Lacroix-Desmazes S. et al., Semin Thromb Hemost. 2000; 26 (2):157), necrotizing small vessel vasculitis, microscopic polyangiitis, Churg and Strauss syndrome, pauci-immune focal necrotizing and crescentic glomerulonephritis (Noel L H. Ann Med Interne (Paris). 2000 May; 151 (3):178), antiphospholipid syndrome (Flamholz R. et al., J Clin Apheresis 1999; 14 (4):171), antibody-induced heart failure (Wallukat G. et al., Am J Cardiol. Jun. 17, 1999; 83 (12A):75H), thrombocytopenic purpura (Moccia F. Ann Ital Med Int. 1999 April-June; 14 (2):114; Semple J W. et al., Blood 1996 May 15; 87 (10):4245), autoimmune hemolytic anemia (Efremov D G. et al., Leuk Lymphoma 1998 January; 28 (3-4):285; Sallah S. et al., Ann Hematol 1997 March; 74 (3):139), cardiac autoimmunity in Chagas' disease (Cunha-Neto E. et al., J Clin Invest Oct. 15, 1996; 98 (8):1709) and anti-helper T lymphocyte autoimmunity (Caporossi A P. et al., Viral Immunol 1998; 11 (1):9).

Examples of autoimmune rheumatoid diseases include, but are not limited to rheumatoid arthritis (Krenn V. et al., Histol Histopathol 2000 July; 15 (3):791; Tisch R, McDevitt H O. Proc Natl Acad Sci units S A Jan. 18, 1994; 91 (2):437) and ankylosing spondylitis (Jan Voswinkel et al., Arthritis Res 2001; 3 (3): 189).

Examples of autoimmune glandular diseases include, but are not limited to, pancreatic disease, Type I diabetes, thyroid disease, Graves' disease, thyroiditis, spontaneous autoimmune thyroiditis, Hashimoto's thyroiditis, idiopathic myxedema, ovarian autoimmunity, autoimmune anti-sperm infertility, autoimmune prostatitis and Type I autoimmune polyglandular syndrome. Diseases include, but are not limited to autoimmune diseases of the pancreas, Type 1 diabetes (Castano L. and Eisenbarth G S. Ann. Rev. Immunol. 8:647; Zimmet P. Diabetes Res Clin Pract 1996 October; 34 Suppl:S125), autoimmune thyroid diseases, Graves' disease (Orgiazzi J. Endocrinol Metab Clin North Am 2000 June; 29 (2):339; Sakata S. et al., Mol Cell Endocrinol 1993 March; 92 (1):77), spontaneous autoimmune thyroiditis (Braley-Mullen H. and Yu S, J Immunol Dec. 15, 2000; 165 (12):7262), Hashimoto's thyroiditis (Toyoda N. et al., Nippon Rinsho 1999 August; 57 (8):1810), idiopathic myxedema (Mitsuma T. Nippon Rinsho. 1999 August; 57 (8):1759), ovarian autoimmunity (Garza K M. et al., J Reprod Immunol 1998 February; 37 (2):87), autoimmune anti-sperm infertility (Diekman A B. et al., Am J Reprod Immunol. 2000 March; 43 (3):134), autoimmune prostatitis (Alexander R B. et al., Urology 1997 December; 50 (6):893) and Type I autoimmune polyglandular syndrome (Hara T. et al., Blood. Mar. 1, 1991; 77 (5):1127).

Examples of autoimmune gastrointestinal diseases include, but are not limited to, chronic inflammatory intestinal diseases (Garcia Herola A. et al., Gastroenterol Hepatol. 2000 January; 23 (1):16), celiac disease (Landau Y E. and Shoenfeld Y. Harefuah Jan. 16, 2000; 138 (2):122), colitis, ileitis and Crohn's disease.

Examples of autoimmune cutaneous diseases include, but are not limited to, autoimmune bullous skin diseases, such as, but are not limited to, Pemphigus vulgaris, bullous pemphigoid and Pemphigus foliaceus.

Examples of autoimmune hepatic diseases include, but are not limited to, hepatitis, autoimmune chronic active hepatitis (Franco A. et al., Clin Immunol Immunopathol 1990 March; 54 (3):382), primary biliary cirrhosis (Jones D E. Clin Sci (Colch) 1996 November; 91 (5):551; Strassburg C P. et al., Eur J Gastroenterol Hepatol. 1999 June; 11 (6):595) and autoimmune hepatitis (Manns M P. J Hepatol 2000 August; 33 (2):326).

Examples of autoimmune neurological diseases include, but are not limited to, multiple sclerosis (Cross A H. et al., J Neuroimmunol Jan. 1, 2001; 112 (1-2):1), Alzheimer's disease (Oron L. et al., J Neural Transm Suppl. 1997; 49:77), myasthenia gravis (Infante A J. And Kraig E, Int Rev Immunol 1999; 18 (1-2):83; Oshima M. et al., Eur J Immunol 1990 December; 20 (12):2563), neuropathies, motor neuropathies (Kornberg A J. J Clin Neurosci. 2000 May; 7 (3):191); Guillain-Barre syndrome and autoimmune neuropathies (Kusunoki S. Am J Med Sci. 2000 April; 319 (4):234), myasthenia, Lambert-Eaton myasthenic syndrome (Takamori M. Am J Med Sci. 2000 April; 319 (4):204); paraneoplastic neurological diseases, cerebellar atrophy, paraneoplastic cerebellar atrophy and stiff-man syndrome (Hiemstra H S. et al., Proc Natl Acad Sci units S A Mar. 27, 2001; 98 (7):3988); non-paraneoplastic stiff man syndrome, progressive cerebellar atrophies, encephalitis, Rasmussen's encephalitis, amyotrophic lateral sclerosis, Sydeham chorea, Gilles de la Tourette syndrome and autoimmune polyendocrinopathies (Antoine J C. and Honnorat J. Rev Neurol (Paris) 2000 January; 156 (1):23); dysimmune neuropathies (Nobile-Orazio E. et al., Electroencephalogr Clin Neurophysiol Suppl 1999; 50:419); acquired neuromyotonia, arthrogryposis multiplex congenita (Vincent A. et al., Ann N Y Acad Sci. 1998 May 13; 841:482), neuritis, optic neuritis (Soderstrom M. et al., J Neurol Neurosurg Psychiatry 1994 May; 57 (5):544) and neurodegenerative diseases.

Examples of autoimmune muscular diseases include, but are not limited to, myositis, autoimmune myositis and primary Sjogren's syndrome (Feist E. et al., Int Arch Allergy Immunol 2000 September; 123 (1):92) and smooth muscle autoimmune disease (Zauli D. et al., Biomed Pharmacother 1999 June; 53 (5-6):234).

Examples of autoimmune nephric diseases include, but are not limited to, nephritis and autoimmune interstitial nephritis (Kelly C J. J Am Soc Nephrol 1990 August; 1 (2):140).

Examples of autoimmune diseases related to reproduction include, but are not limited to, repeated fetal loss (Tincani A. et al., Lupus 1998; 7 Suppl 2:S107-9).

Examples of autoimmune connective tissue diseases include, but are not limited to, ear diseases, autoimmune ear diseases (Yoo T J. et al., Cell Immunol 1994 August; 157 (1):249) and autoimmune diseases of the inner ear (Gloddek B. et al., Ann N Y Acad Sci Dec. 29, 1997; 830:266).

Examples of autoimmune systemic diseases include, but are not limited to, systemic lupus erythematosus (Erikson J. et al., Immunol Res 1998; 17 (1-2):49) and systemic sclerosis (Renaudineau Y. et al., Clin Diagn Lab Immunol. 1999 March; 6 (2):156); Chan O T. et al., Immunol Rev 1999 June; 169:107).

Neurodegenerative Diseases:

Examples of neurodegenerative diseases include, but are not limited to, Alexander disease, Alper's disease, Alzheimer's disease, Amyotrophic lateral sclerosis, Ataxia telangiectasia, Batten disease (also known as Spielmeyer-Vogt-Sjogren-Batten disease), Bovine spongiform encephalopathy (BSE), Canavan disease, Cockayne syndrome, Corticobasal degeneration, Creutzfeldt-Jakob disease, Huntington disease, HIV-associated dementia, Kennedy's disease, Krabbe disease, Lewy body dementia, Machado-Joseph disease (Spinocerebellar ataxia type 3), Multiple sclerosis, Multiple System Atrophy, Neuroborreliosis, Parkinson disease, Pelizaeus-Merzbacher Disease, Pick's disease, Primary lateral sclerosis, Prion diseases, Refsum's disease, Sandhoff disease, Schilder's disease, Sub-Acute Combined Degeneration of the Cord Secondary to Pernicious Anaemia, Schizophrenia, Spielmeyer-Vogt-Sjogren-Batten disease (also known as Batten disease), Spinocerebellar ataxia (multiple types with varying characteristics), Spinal muscular atrophy, Steele-Richardson-Olszewski disease and Tabes dorsalis.

Infectious Diseases

Examples of infectious diseases include, but are not limited to, chronic infectious diseases, subacute infectious diseases, acute infectious diseases, viral diseases, bacterial diseases, protozoan diseases, parasitic diseases, fungal diseases, mycoplasma diseases and prion diseases.

Viral diseases which may be treated according to embodiments of the present invention are those caused by pathogenic viruses belonging to the following families: Adenoviridae, Coronaviridae, Picornaviridae, Herpesviridae, Hepadnaviridae, Flaviviridae, Retroviridae, Orthomyxoviridae, Paramyxoviridae, Papovaviridae, Polyomavirus, Rhabdoviridae and Togaviridae.

Particular pathogenic viruses contemplated by the present invention are those that cause smallpox, influenza, mumps, measles, chickenpox, ebola, or rubella.

According to a particular embodiment, the virus is one which brings about a respiratory infection (e.g. an upper respiratory tract infection and/or a lower respiratory tract infection).

Thus, according to a particular embodiment, the pathogenic virus is an influenza virus (e.g. influenza virus A—(e.g. H1N1, H2N2, H3N2, H5N1, H7N7, H1N2, H9N2, H7N2, H7N3, H10N7 and H7N9), influenza virus B or influenza virus C).

In another embodiment, the pathogenic virus is a coronavirus e.g. COVID-19.

In another embodiment, the pathogenic virus is a parainfluenza virus (hPIV) including the human parainfluenza virus type 1 (hPIV-1) (causes croup); the human parainfluenza virus type 2 (hPIV-2) (causes croup and other upper and lower respiratory tract illnesses), the human parainfluenza virus type 3 (hPIV-3) (associated with bronchiolitis and pneumonia) and the human parainfluenza virus type 4 (hPIV-4).

In yet another embodiment, the pathogenic virus is a respiratory syncytial virus (RSV).

The pathogenic bacteria may be gram positive or gram negative bacteria.

Exemplary pathogenic bacteria include Mycobacterium tuberculosis which causes tuberculosis, Streptococcus and Pseudomonas which cause pneumonia, and Shigella, Campylobacter and Salmonella which cause foodborne illnesses. Other exemplary pathogenic bacteria contemplated by the present invention are those that cause infections such as tetanus, typhoid fever, diphtheria, syphilis and Hansen's disease.

According to a particular embodiment, the pathogenic bacteria is E. coli, Klebsiella pneumonia, Enterococcus faecalis, Staphylococcus aureus (MSSA, MRSA), Salmonella enteritidis or Serratia marcescens.

According to one embodiment, the infection is an acute infection.

According to another embodiment, the infection is a chronic infection.

In particular embodiments, the subject which is treated for an infectious disease shows symptoms of an infection—e.g. has a fever.

According to a particular embodiment, the infectious disease is conjunctivitis (e.g. viral conjunctivitis).

According to still another embodiment, the disease is a cardiovascular disease.

According to still another embodiment, the disease is triple negative breast cancer.

According to still another embodiment, the disease is head and neck cancer.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

As used herein, the phrase “subject in need thereof” refers to a subject which has the disease. The subject may be a mammal, e.g. a human.

The agents may be provided per se or as part of a pharmaceutical composition.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the agents which bind (and preferably activate CD45) accountable for the biological effect. In another embodiment, the active ingredient is the activated T cells (as described herein).

Hereinafter, the phrases “physiologically acceptable carrier” and “pharmaceutically acceptable carrier” which may be interchangeably used refer to a carrier or a diluent that does not abrogate the biological activity and properties of the administered compound. The carrier may also include biological or chemical substances that modulate the immune response.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intracardiac, e.g., into the right or left ventricular cavity, into the common coronary artery, intravenous, intraperitoneal, intranasal, or intraocular injections.

Conventional approaches for drug delivery to the central nervous system (CNS) include: neurosurgical strategies (e.g., intracerebral injection or intracerebroventricular infusion); molecular manipulation of the agent (e.g., production of a chimeric fusion protein that comprises a transport peptide that has an affinity for an endothelial cell surface molecule in combination with an agent that is itself incapable of crossing the BBB) in an attempt to exploit one of the endogenous transport pathways of the BBB; pharmacological strategies designed to increase the lipid solubility of an agent (e.g., conjugation of water-soluble agents to lipid or cholesterol carriers); and the transitory disruption of the integrity of the BBB by hyperosmotic disruption (resulting from the infusion of a mannitol solution into the carotid artery or the use of a biologically active agent such as an angiotensin peptide). However, each of these strategies has limitations, such as the inherent risks associated with an invasive surgical procedure, a size limitation imposed by a limitation inherent in the endogenous transport systems, potentially undesirable biological side effects associated with the systemic administration of a chimeric molecule comprised of a carrier motif that could be active outside of the CNS, and the possible risk of brain damage within regions of the brain where the BBB is disrupted, which renders it a suboptimal delivery method.

Alternately, one may administer the pharmaceutical composition in a local rather than systemic manner, for example, via injection of the pharmaceutical composition directly into a tissue region of a patient.

The term “tissue” refers to part of an organism consisting of cells designed to perform a function or functions. Examples include, but are not limited to, brain tissue, retina, skin tissue, hepatic tissue, pancreatic tissue, bone, cartilage, connective tissue, blood tissue, muscle tissue, cardiac tissue brain tissue, vascular tissue, renal tissue, pulmonary tissue, gonadal tissue, hematopoietic tissue.

Pharmaceutical compositions of some embodiments of the invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with some embodiments of the invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to some embodiments of the invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of some embodiments of the invention may also be formulated in rectal compositions such as suppositories or retention enemas, using, e.g., conventional suppository bases such as cocoa butter or other glycerides.

Pharmaceutical compositions suitable for use in context of some embodiments of the invention include compositions wherein the active ingredients are contained in an amount effective to achieve the intended purpose. More specifically, a therapeutically effective amount means an amount of active ingredients effective to prevent, alleviate or ameliorate symptoms of a disorder or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p.1).

Dosage amount and interval may be adjusted individually to provide tissue levels of the active ingredient are sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions of some embodiments of the invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

The present inventors propose that the agents capable of binding (and preferably activating CD45) described herein will be synergistic with additional agents which are therapeutic for cancer. Thus, the present inventors contemplate administering the CD45 binding agents with a chemotherapeutic agent (for example those that do not have a mechanism of action which includes activating CD45) for the treatment of cancer.

Examples of chemotherapeutic agents include, but are not limited to Acivicin; Aclarubicin; Acodazole Hydrochloride; Acronine; Adriamycin; Adozelesin; Aldesleukin; Altretamine; Ambomycin; Ametantrone Acetate; Aminoglutethimide; Amsacrine; Anastrozole; Anthramycin; Asparaginase; Asperlin; Azacitidine; Azetepa; Azotomycin; Batimastat; Benzodepa; Bicalutamide; Bisantrene Hydrochloride; Bisnafide Dimesylate; Bizelesin; Bleomycin Sulfate; Brequinar Sodium; Bropirimine; Busulfan; Cactinomycin; Calusterone; Caracemide; Carbetimer; Carboplatin; Carmustine; Carubicin Hydrochloride; Carzelesin; Cedefingol; Chlorambucil; Cirolemycin; Cisplatin; Cladribine; Crisnatol Mesylate; Cyclophosphamide; Cytarabine; Dacarbazine; Dactinomycin; Daunorubicin Hydrochloride; Decitabine; Dexormaplatin; Dezaguanine; Dezaguanine Mesylate; Diaziquone; Docetaxel; Doxorubicin; Doxorubicin Hydrochloride; Droloxifene; Droloxifene Citrate; Dromostanolone Propionate; Duazomycin; Edatrexate; Eflornithine Hydrochloride; Elsamitrucin; Enloplatin; Enpromate; Epipropidine; Epirubicin Hydrochloride; Erbulozole; Esorubicin Hydrochloride; Estramustine; Estramustine Phosphate Sodium; Etanidazole; Etoposide; Etoposide Phosphate; Etoprine; Fadrozole Hydrochloride; Fazarabine; Fenretinide; Floxuridine; Fludarabine Phosphate; Fluorouracil; Flurocitabine; Fosquidone; Fostriecin Sodium; Gemcitabine; Gemcitabine Hydrochloride; Hydroxyurea; Idarubicin Hydrochloride; Ifosfamide; Ilmofosine; Interferon Alfa-2a; Interferon Alfa-2b; Interferon Alfa-n1; Interferon Alfa-n3; Interferon Beta-I a; Interferon Gamma-I b; Iproplatin; Irinotecan Hydrochloride; Lanreotide Acetate; Letrozole; Leuprolide Acetate; Liarozole Hydrochloride; Lometrexol Sodium; Lomustine; Losoxantrone Hydrochloride; Masoprocol; Maytansine; Mechlorethamine Hydrochloride; Megestrol Acetate; Melengestrol Acetate; Melphalan; Menogaril; Mercaptopurine; Methotrexate; Methotrexate Sodium; Metoprine; Meturedepa; Mitindomide; Mitocarcin; Mitocromin; Mitogillin; Mitomalcin; Mitomycin; Mitosper; Mitotane; Mitoxantrone Hydrochloride; Mycophenolic Acid; Nocodazole; Nogalamycin; Ormaplatin; Oxisuran; Paclitaxel; Oxaliplatin; Pegaspargase; Peliomycin; Pentamustine; Peplomycin Sulfate; Perfosfamide; Pipobroman; Piposulfan; Piroxantrone Hydrochloride; Plicamycin; Plomestane; Porfimer Sodium; Porfiromycin; Prednimustine; Procarbazine Hydrochloride; Puromycin; Puromycin Hydrochloride; Pyrazofurin; Riboprine; Rogletimide; Safingol; Safingol Hydrochloride; Semustine; Simtrazene; Sparfosate Sodium; Sparsomycin; Spirogermanium Hydrochloride; Spiromustine; Spiroplatin; Streptonigrin; Streptozocin; Sulofenur; Talisomycin; Taxol; Tecogalan Sodium; Tegafur; Teloxantrone Hydrochloride; Temoporfin; Teniposide; Teroxirone; Testolactone; Thiamiprine; Thioguanine; Thiotepa; Tiazofuirin; Tirapazamine; Topotecan Hydrochloride; Toremifene Citrate; Trestolone Acetate; Triciribine Phosphate; Trimetrexate; Trimetrexate Glucuronate; Triptorelin; Tubulozole Hydrochloride; Uracil Mustard; Uredepa; Vapreotide; Verteporfin; Vinblastine Sulfate; Vincristine Sulfate; Vindesine; Vindesine Sulfate; Vinepidine Sulfate; Vinglycinate Sulfate; Vinleurosine Sulfate; Vinorelbine Tartrate; Vinrosidine Sulfate; Vinzolidine Sulfate; Vorozole; Zeniplatin; Zinostatin; Zorubicin Hydrochloride. Additional antineoplastic agents include those disclosed in Chapter 52, Antineoplastic Agents (Paul Calabresi and Bruce A. Chabner), and the introduction thereto, 1202-1263, of Goodman and Gilman's “The Pharmacological Basis of Therapeutics”, Eighth Edition, 1990, McGraw-Hill, Inc. (Health Professions Division).

According to a particular embodiment, the chemotherapeutic agent is an immune checkpoint inhibitor.

As used herein, the phrase “immune checkpoint inhibitor” refers to a compound capable of inhibiting the function of an immune checkpoint protein. Inhibition includes reduction of function and full blockade. In particular the immune checkpoint protein is a human immune checkpoint protein. Thus the immune checkpoint protein inhibitor preferably is an inhibitor of a human immune checkpoint protein. Immune checkpoint proteins are described in the art (see for instance Pardoll, 2012. Nature Rev. cancer 12: 252-264). The designation immune checkpoint includes the experimental demonstration of stimulation of an antigen-receptor triggered T lymphocyte response by inhibition of the immune checkpoint protein in vitro or in vivo, e.g. mice deficient in expression of the immune checkpoint protein demonstrate enhanced antigen-specific T lymphocyte responses or signs of autoimmunity (such as disclosed in Waterhouse et al., 1995. Science 270:985-988; Nishimura et al., 1999. Immunity 11:141-151). It may also include demonstration of inhibition of antigen-receptor triggered CD4+ or CD8+ T cell responses due to deliberate stimulation of the immune checkpoint protein in vitro or in vivo (e.g. Zhu et al., 2005. Nature Immunol. 6:1245-1252).

Exemplary immune checkpoint protein inhibitors are antibodies that specifically recognize immune checkpoint proteins. A number of CTLA-4, PD1, PDL-1, PD-L2, LAG-3, BTLA, B7H3, B7H4, TIM3 and KIR inhibitors are known and in analogy of these known immune checkpoint protein inhibitors, alternative immune checkpoint inhibitors may be developed in the (near) future. For example ipilimumab is a fully human CTLA-4 blocking antibody presently marketed under the name Yervoy (Bristol-Myers Squibb). A second CTLA-4 inhibitor is tremelimumab (referenced in Ribas et al, 2013, J. Clin. Oncol. 31:616-22). Examples of PD-1 inhibitors include without limitation humanized antibodies blocking human PD-1 such as lambrolizumab (e.g. disclosed as hPD109A and its humanized derivatives h409A11, h409A16 and h409A17 in WO2008/156712; Hamid et al., N. Engl. J. Med. 369: 134-144 2013), or pidilizumab (disclosed in Rosenblatt et al., 2011. J. Immunother. 34:409-18), as well as fully human antibodies such as nivolumab (previously known as MDX-1106 or BMS-936558, Topalian et al., 2012. N. Eng. J. Med. 366:2443-2454, disclosed in U.S. Pat. No. 8,008,449 B2). Other PD-1 inhibitors may include presentations of soluble PD-1 ligand including without limitation PD-L2 Fc fusion protein also known as B7-DC-Ig or AMP-244 (disclosed in Mkrtichyan M, et al. J Immunol. 189:2338-47 2012) and other PD-1 inhibitors presently under investigation and/or development for use in therapy. In addition, immune checkpoint inhibitors may include without limitation humanized or fully human antibodies blocking PD-L such as MEDI-4736 (disclosed in WO2011066389 A1), MPDL3280A (disclosed in U.S. Pat. No. 8,217,149 B2) and MIH1 (Affymetrix obtainable via eBioscience (16.5983.82)) and other PD-L1 inhibitors presently under investigation. According to this invention an immune checkpoint inhibitor is preferably selected from a CTLA-4, PD-1 or PD-L1 inhibitor, such as selected from the known CTLA-4, PD-1 or PD-L1 inhibitors mentioned above (ipilimumab, tremelimumab, labrolizumab, nivolumab, pidilizumab, AMP-244, MEDI-4736, MPDL3280A, MIH1). Known inhibitors of these immune checkpoint proteins may be used as such or analogues may be used, in particular chimerized, humanized or human forms of antibodies.

In a particular embodiment, the chemotherapeutic agent is not a nonmyeloablative lymphodepleting chemotherapy such as cyclophosphamide and fludarabine.

In the context of a combination therapy, the chemotherapeutic agent may be administered by the same route of administration (e.g. intrapulmonary, oral, enteral, etc.) as the CD45 binding agent is administered. In the alternative, the chemotherapeutic agent may be administered by a different route of administration to the CD45 binding agent.

The chemotherapeutic agent can be administered immediately prior to (or after) the CD45 binding agent, on the same day as, one day before (or after), one week before (or after), one month before (or after), or two months before (or after) the CD45 binding agent, and the like.

The chemotherapeutic agent and the CD45 binding agent can be administered concomitantly, that is, where the administering for each of these reagents can occur at time intervals that partially or fully overlap each other. The chemotherapeutic agent and the CD45 binding agent can be administered during time intervals that do not overlap each other. For example, the chemotherapeutic agent can be administered within the time frame of t=0 to 1 hours, while the CD45 binding agent can be administered within the time frame of t=1 to 2 hours. Also, the chemotherapeutic agent can be administered within the time frame of t=0 to 1 hours, while the CD45 binding agent can be administered somewhere within the time frame of t=2-3 hours, t=3-4 hours, t=4-5 hours, t=5-6 hours, t=6-7 hours, t=7-8 hours, t=8-9 hours, t=9-10 hours, and the like. Moreover, the CD45 binding agent can be administered somewhere in the time frame of t=minus 2-3 hours, t=minus 3-4 hours, t=minus 4-5 hours, t=5-6 minus hours, t=minus 6-7 hours, t=minus 7-8 hours, t=minus 8-9 hours, t=minus 9-10 hours.

The CD45 binding agent of the present invention and the chemotherapeutic agent are typically provided in combined amounts to achieve therapeutic, prophylactic and/or pain palliative effectiveness. This amount will evidently depend upon the particular compound selected for use, the nature and number of the other treatment modality, the condition(s) to be treated, prevented and/or palliated, the species, age, sex, weight, health and prognosis of the subject, the mode of administration, effectiveness of targeting, residence time, mode of clearance, type and severity of side effects of the pharmaceutical composition and upon many other factors which will be evident to those of skill in the art. The CD45 binding agent will be used at a level at which therapeutic, prophylactic and/or pain palliating effectiveness in combination with the chemotherapeutic agent is observed.

The chemotherapeutic agent may be administered (together with the CD45 binding agent) at a gold standard dosing as a single agent, below a gold standard dosing as a single agent or above a gold standard dosing as a single agent.

According to specific embodiments, the chemotherapeutic agent is administered below the gold standard dosing as a single agent.

As used herein the term “gold standard dosing” refers to the dosing which is recommended by a regulatory agency (e.g., FDA), for a given tumor at a given stage.

According to other specific embodiments, the chemotherapeutic agent is administered at a dose that does not exert at least one side effect which is associated with the gold standard dosing. Non-limiting examples of side effects of a chemotherapeutic agent treatment include skin rash, diarrhea, mouth sores, paronychia, fatigue, hyperglycemia, hepatotoxicity, kidney failure, cardiovascular effects, electrolytes anomalies and GI perforations.

Thus, in one embodiment, the amount of the chemotherapeutic agent is below the minimum dose required for therapeutic, prophylactic and/or pain palliative effectiveness when used as a single therapy (e.g. 10-99%, preferably 25 to 75% of that minimum dose). This allows for reduction of the side effects caused by the chemotherapeutic agent but the therapy is rendered effective because in combination with the CD45 binding agent, the combinations are effective overall.

In one aspect of the present invention, the CD45 binding agent and the chemotherapeutic agent are synergistic with respect to their dosages. That is to say that the effect provided by the CD45 binding agent of the present invention is greater than would be anticipated from the additive effects of the chemotherapeutic agent and the CD45 binding agent when used separately. In an alternative embodiment, the chemotherapeutic agent of the present invention and the CD45 binding agent are synergistic with respect to their side effects. That is to say that the side-effects caused by the CD45 binding agent in combination with the chemotherapeutic agent are less than would be anticipated when the equivalent therapeutic effect is provided by either the chemotherapeutic agent or CD45 binding agent when used separately.

Ex Vivo Treatment

According to another aspect of the present invention, there is provided a method of activating T cells (e.g. increasing the cytotoxicity of T cells), the method comprising incubating T cells with pathogenic cells in the presence of an agent that binds to CD45 of said T cells, under conditions which allow expansion of said T cells, with the proviso that said agent is not a multimeric peptide comprising at least two peptide monomers linked to one another, each of said at least two peptide monomers comprising at least 6 consecutive amino acids from the amino acid sequence as set forth in SEQ ID NO: 1, wherein said at least two peptide monomers are each no longer than 30 amino acids.

Agents that Bind to CD45

In one embodiment, the agent is a CD45 agonist.

In another embodiment, the agent is capable of at least one of the following:

(i) decrease the phosphorylation status of Lck at position 505 in T cells and/or NK cells;

(ii) increase the phosphorylation status of Lck at position 394 in T cells and/or NK cells;

(iii) increase the phosphorylation status of VAV-1 in T cells and/or NK cells.

(iv) increase the phosphorylation status of ZAP-70 at position 493 in T cells and/or NK cells.

In still another embodiment, the agent is an antibody which binds to CD45 (e.g. an activating antibody—see for example US Application No. 20190359713).

T Cells

The term “T cells” refers to cytotoxic T cells. Cytotoxic T cells typically express a T cell receptor that binds to a specific antigen on the target cell.

The T cells may be derived from any mammalian species, such as human and may be obtained from white blood cell preparations or peripheral blood mononuclear cells (PBMCs) derived from a subject.

Alternatively, the T cells may have migrated into the tumor (i.e. may be comprised in tumor-infiltrating lymphocytes). Tumor infiltrating lymphocytes (TILs) can be isolated from an individual (e.g. during a tumor biopsy) and cultured in vitro (Kawakami, Y. et al. (1989) J. Immunol. 142: 2453-3461). An exemplary method for obtaining TILs includes plating viable cells (e.g. 1×10⁶) of a single-cell suspension of enzymatically digested explant of metastatic tumor. It will be appreciated that the TILs may be isolated from fresh tumors or from frozen tissue (at the cost of lower yield).

The T cells are typically comprised in a heterogeneous population of white blood cells which includes antigen presenting cells and macrophages.

As mentioned, the T cells may be derived from PBMCs. PBMCs may be prepared as follows: Buffy coats from blood bank donors are layered onto Lymphoprep solution (Nycomed, Oslo, Norway) and spun at 2000 rpm for about 20 minutes. The interface layer is collected, washed, counted, and resuspended in PBS; pH 7.4 to the desired cell concentration.

The T-cells may be modified to express an antibody or a T-cell growth factor that promotes the growth and activation thereof. The T cells may comprise a chimeric antigen receptor (i.e. Car-T cells). Any suitable methods of modification may be used. See, e.g., Sambrook and Russell, Molecular Cloning, 3^{rd} ed., SCHL Press (2001). Desirably, modified T-cells express the T-cell growth factor at high levels. T-cell growth factor coding sequences, such as that of IL-2, are readily available in the art, as are promoters, the operable linkage of which to a T-cell growth factor coding sequence promote high-level expression.

Pathogenic Cells:

Contemplated pathogenic cells include any cells that comprise antigenic determinants on their surface. The pathogenic cells may be bacterial, fungal or viral. The pathogenic cells may be cells damaged by environmental factors such as ultraviolet light and other radiations. Alternatively, the pathogenic cells may be diseased cells such as cancer cells.

The pathogenic cells may be obtained from a patient (e.g. during a biopsy) or may be available as a cell line.

Incubation Reaction

As mentioned, the method of this aspect of the present invention comprises incubating the white blood cell population (which comprises T cells and antigen presenting cells) with pathogenic cells in the presence of an agent that binds to CD45 under conditions which allow activation and expansion of the T cells.

The phrase “activation of the T cells” refers to the induction of a cytotoxic activity in the T cells.

Preferably, the white blood cell population is incubated with the pathogenic cells and the agent that binds to CD45 for at least one day, more preferably at least two days, three days, four days, five days, six days, seven days or more so as to ensure activation.

Additional agents may be included in the incubation including for example serum (e.g. fetal calf serum) or serum replacements. The agent that binds to CD45 may be added throughout the incubation period or at one or two day intervals.

Preferably, the cells are cultured together with the agent that binds to CD45 under conditions that ensure survival or propagation of the T cells. Such conditions include incubating at appropriate temperatures and pressure and in a medium that ensures cell survival. Exemplary media include RPMI or RPMI 1640 or AIM V.

The present invention contemplates expanding the T cells concomitantly with the activation and/or following the activation.

Expansion of T-cell cultures can be accomplished by any of a number of methods as are known in the arts. For example, T cells may be expanded utilizing non-specific T-cell receptor stimulation in the presence of feeder lymphocytes and either IL-2 or IL-15. The non-specific T-cell receptor stimulus can consist of around 30 ng/ml of OKT3, a mouse monoclonal anti-CD3 antibody available from Ortho, Raritan, N.J.

Following generation of cytotoxic T cells, they may be isolated to generate a homogeneous population of isolated cytotoxic T cells.

Methods of isolating cytotoxic T cells from a mixed population of cells are known in the art and include for example isolating T cells based on the expression of a cell surface antigens such as CD8. This may be performed using flow cytometry. A multitude of flow cytometers are commercially available including for e.g. Becton Dickinson FACScan and FACScaliber (BD Biosciences, Mountain View, Calif.). Antibodies that may be used for FACS analysis are taught in Schlossman S, Boumell L, et al, [Leucocyte Typing V. New York: Oxford University Press; 1995] and are widely commercially available.

Additionally, or alternatively, a substrate including an antibody or a ligand capable of specifically binding cell surface markers present on “harmful” or non-relevant cells, can be used to effectively deplete these cells from the mixed population of cells.

The affinity substrate according to the present invention can be a column matrix such as, for example agarose, cellulose and the like, or beads such as, for example, magnetic beads onto which the antibodies described above, are immobilized.

Using the methods described above cytotoxic T cells and T cell lines may be obtained.

Thus, according to another aspect of the present invention there is provided a cytotoxic T cell line which comprises an agent that binds to CD45 which is present on T cells of the T cell line.

The present invention contemplates a T cell line wherein the agent that binds to CD45 described herein above binds to at least 5% of the cells, at least 10% of the cells, at least 15% of the cells, at least 20% of the cells, at least 25% of the cells, at least 30% of the cells, at least 35% of the cells, at least 40% of the cells, at least 45% of the cells, at least 50% of the cells, at least 55% of the cells, at least 60% of the cells, at least 65% of the cells, at least 70% of the cells, at least 75% of the cells, at least 80% of the cells, at least 85% of the cells, at least 90% of the cells, at least 95% of the cells, at least 99% of the cells, or even to 100% of the cells.

Exemplary methods of assaying activities of T cell lines include ⁵¹CR release cytotoxicity assays (Cerundolo, V. et al. (1990) Nature 345:449-452) or lymphokine assays such as IFN-γ or TNF secretion assays [Schwartzentruber, D. et al., (1991) J. of Immunology 146:3674-3681].

The T cell lines described herein may be used for treating subjects having diseases which are amenable to treatment by adoptive immunotherapy (e.g. cancer, autoimmune diseases, HIV, hepatitis, HHV6, chronic fatigue syndrome).

The T cell lines may be used immediately following generation or may be stored (e.g. frozen) and used when needed.

Exemplary cancers which may be treated using the T cell lines described herein include, but are not limited to, adrenocortical carcinoma, hereditary; bladder cancer; breast cancer; breast cancer, ductal; breast cancer, invasive intraductal; breast cancer, sporadic; breast cancer, susceptibility to; breast cancer, type 4; breast cancer, type 4; breast cancer-1; breast cancer-3; breast-ovarian cancer; Burkitt's lymphoma; cervical carcinoma; colorectal adenoma; colorectal cancer; colorectal cancer, hereditary nonpolyposis, type 1; colorectal cancer, hereditary nonpolyposis, type 2; colorectal cancer, hereditary nonpolyposis, type 3; colorectal cancer, hereditary nonpolyposis, type 6; colorectal cancer, hereditary nonpolyposis, type 7; dermatofibrosarcoma protuberans; endometrial carcinoma; esophageal cancer; gastric cancer, fibrosarcoma, glioblastoma multiforme; Glomus tumors, multiple; hepatoblastoma; hepatocellular cancer; hepatocellular carcinoma; leukemia, acute lymphoblastic; leukemia, acute myeloid; leukemia, acute myeloid, with eosinophilia; leukemia, acute nonlymphocytic; leukemia, chronic myeloid; Li-Fraumeni syndrome; liposarcoma, lung cancer; lung cancer, small cell; lymphoma, non-Hodgkin's; lynch cancer family syndrome II; male germ cell tumor; mast cell leukemia; medullary thyroid; medulloblastoma; melanoma, meningioma; multiple endocrine neoplasia; myeloid malignancy, predisposition to; myxosarcoma, neuroblastoma; osteosarcoma; ovarian cancer; ovarian cancer, serous; ovarian carcinoma; ovarian sex cord tumors; pancreatic cancer; pancreatic endocrine tumors; paraganglioma, familial nonchromaffin; pilomatricoma; pituitary tumor, invasive; prostate adenocarcinoma; prostate cancer; renal cell carcinoma, papillary, familial and sporadic; retinoblastoma; rhabdoid predisposition syndrome, familial; rhabdoid tumors; rhabdomyosarcoma; small-cell cancer of lung; soft tissue sarcoma, squamous cell carcinoma, head and neck; T-cell acute lymphoblastic leukemia; Turcot syndrome with glioblastoma; tylosis with esophageal cancer; uterine cervix carcinoma, Wilms' tumor, type 2; and Wilms' tumor, type 1, etc.

Preferably, the cancer is breast cancer, melanoma, lung carcinoma, colon cancer, prostate cancer, ovarian carcinoma, renal cell carcinoma, glioma, Hodgkin's lymphoma, non-Hodgkin's lymphoma, acute lymphatic leukemia (ALL) and the like. The cancer may be metastatic or non-metastatic.

According to a particular embodiment, the cancer is breast cancer (e.g. triple negative breast cancer).

According to still another embodiment, the cancer is a head and neck cancer.

It will be appreciated that preparation of cytotoxic T cell lines for treatment of a disease in a particular subject may be effected using components which are autologous to that subject. Thus, for example, the present invention contemplates using T cells retrieved from the patient for generating the T cell line. Additionally and/or alternatively the pathogenic cells used to stimulate the T cells may be autologous to the subject.

The present inventors have shown that as long as the pathogenic cells used to activate the T cells share at least one HLA class I allele with the pathogenic cells present in the subject the generated T cell lines will be cytotoxic and effective at treating the disease in the subject.

Thus, the pathogenic cells used to stimulate the T cells are preferably allogeneic with the pathogenic cells in the subject. Verdegaal et al., Human Immunology 60, 1196-1206, 1999, the contents of which are incorporated by reference herein teaches various tumors which share HLA class I alleles.

Thus, the present invention contemplates activating T cells with breast cancer cells and using the activated T cells for treating renal cell carcinoma, colon cancer, renal cancer and/or melanoma.

In addition, the present invention contemplates activating T cells with one type of breast cancer cells and using the activated T cells for treating another type of breast cancer (as long as the cancers share HLA class I alleles).

As mentioned, the present inventors have shown that therapeutic agents that bind to CD45 causes a change in the phosphorylation status of intracellular downstream effectors. The phosphorylation status of these effectors can be used in order to monitor the efficacy of such therapeutic agents.

Thus, according to another aspect of the present invention there is provided a method of monitoring the efficacy of a therapeutic agent that increases the cytotoxicity of T cells by binding to CD45 in a subject, the method comprising analyzing in the T cells of the subject the phosphorylation status of at least one protein selected from the group consisting of Lck, ZAP70 and VAV-1, wherein a change in the phosphorylation status of said at least one protein in the presence of said agent as compared to the phosphorylation status in the absence of said agent is indicative of an efficacious therapeutic agent.

The amino acid sequence of Lck is set forth in SEQ ID NO: 104.

The amino acid sequence of ZAP70 is set forth in SEQ ID NO: 105.

The amino acid sequence of VAV1 is set forth in SEQ ID NO: 106.

Phosphorylation status of the above described proteins can be measured using antibodies specific for the phosphorylated or non/phosphorylated form. Contemplated techniques using the antibodies include Western blots and immunoprecipitation.

As used herein the term “about” refers to ±10%.

The terms “comprises”, “comprising”, “includes”, “including”, “having” and their conjugates mean “including but not limited to”.

The term “consisting of” means “including and limited to”.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

Materials and Methods

The following peptides were synthesized:

| | Name | Lot N | Purity | quantity | MW |
|---|---|---|---|---|---|
| 1. | C24D | JT-62597 | 97% | 50 mg | 6481.82 |
| | (as illustrated in | | | | |
| | FIG. 1) | | | | |
| 2. | C24DLys[Biotin] | JT-62601 | 97% | 15 mg | 7190.81 |
| 3. | [Biotin]-C24D | JT-62599 | 97% | 15 mg | 6934.44 |
| 4. | C24D-Lys[FITC] | JT-62600 | 97% | 15 mg | 7516.94 |

Breast Cancer Cell Lines:

MCF7, estrogen positive. ATCC Number: HTB-22™.

MDAMB468, triple negative, less aggressive. ATCC Number: HTB-132™.

MDAMB231, very aggressive triple negative. ATCC Number: HTB-26™.

Normal breast cell line: MCF10A. ATCC Number: CRL-10317™.

MCF7, MDAMB468 and MDAMB231 breast cancer cell culture: MCF7, MDAMB468 and MDAMB231 human breast carcinoma cells were maintained in monolayer cultures in DMEM medium (Biological Industries, Israel) supplemented with 10% fetal bovine serum (FBS, Invitrogen, Gibco), 1% penicillin/streptomycin, and sodium 1 mM pyruvate (growth medium).

For different cell passages, confluent monolayer cultures were trypsinized with trypsin 0.25% EDTA 0.05% solution (Biological Industries), washed once in DMEM medium, and seeded in growth medium. MCF10A cells (normal breast cells) were used as control.

Isolation of PBMCs form leucocyte-enriched buffy coats: Blood from healthy female donors was obtained from the Blood Bank (Magen David Adom, Tel HaShomer). Blood was provided as buffy coats.

Peripheral blood mononuclear cells (PBMC) were isolated from buffy coats by density gradient centrifugation, using Ficoll-Paque Premium 1.073 (SIGMA).

After leucocyte isolation, the cells were re-suspended in medium RPMI+10% human AB serum for in-vitro experiments.

Patient-derived tumor primary culture medium: Medium D: DMEM/F12 (1:1, v/v) supplemented with 10% FBS and 1% antibiotics/antimycotic (100×. Medium M: Medium 171 supplemented with 1% MEGS (100×), and 1% antibiotics/antimycotics. Medium DB: mixed from medium D and medium M (1:1, v/v).

Tumor biopsies, all from different patients, were collected after obtaining the patients' consent and were transported to the laboratory. Excess adipose tissue was pared off the tumor samples, following which the samples were sliced into small fragments (approximately 1-2 mm²) by using a scalpel. The fragments were seeded per 10 cm² Petri dishes in DB medium. The Petri dishes were incubated at 37° C. with 5% CO₂ and monitored daily to record the time of cell appearance and the cell morphology. The medium was replaced every 3 days. Approximately two weeks after primary culture incubation, cells were trypsinized and cultured in Petri dishes according to the below protocol.

### A. Protocol for the Assessment of C24D Activity:

Day 1: 4×10⁵ tumor cells from the cell line or tumor and stroma cells derived from patient's biopsies were incubated in the respective growth medium in 6 well plates overnight or Petri dishes.
Day 2: 3 hours prior to addition of PBMC of healthy female donors or from autologous patients, DMEM medium was changed to RPMI medium. PBMC was added to the tumor cells (2×10⁶/ml) and C24D was immediately added at 10 μg/ml and incubated for 3 to 6 days, at 37° C., 5% CO₂.
RPMI supplemented Media (1 ml)+C24D was added to the cells every 48 hrs.
Day 3-6: At the end of the experiments, lymphocytes were extracted, centrifuged and re-suspended in PBS supplemented with 0.1% Na Azide and 5% FBS for FACS analysis. Supernatant recovered from lymphocytes centrifugation was stored at −80° C. for interferon gamma determination, tumor cells were washed or trypsinized for tumor cell density or apoptosis assessment.

Tumor cell density: Tumor cells were washed once with PBS and cell density was documented by inverted microscopy (image magnification ×4).

Tumor cell apoptosis analysis: Tumor cells were washed once with PBS and trypsinized for apoptosis analysis using AnnexinV/PI kit, following manufacturer instructions (MEBCYTO-Apoptosis kit, MBL).

Interferon gamma secretion: Supernatants obtained 4-6 days after tumor cell incubation with lymphocytes and peptides were used for Interferon gamma secretion using Human Interferon gamma ELISA Ready SET-Go. eBioscience, following manufacturer instructions.

### B. Protocol for the Determination of C24D Binding to Leucocytes Sub-Populations.

For the determination of the C24D binding to PBMC sub-populations, FACS analysis was performed using a FITC labeled peptide.

Briefly, fresh isolated PBMC from healthy female donors and from breast cancer patients (0.5×10⁶/50 μl PBS+Na Azide+5% FBS) were incubated with the following antibodies: CD3-PC5.5, CD4-PC7, CD8-KO, CD56-PE, CD16-APC, CD14 KO, CD45RA-AF750, CD45RO-PC5.5 and NKG2D-AF750 (Beckman Coulter) and with the addition of 5 μg/5 μg/5 μl C24D-FITC peptide for 40 minutes at room temperature. Next, cells were washed twice (10 minutes, 1200 rpm, 4° C.). Gated live cells were analyzed using Coulter Navious FACS, Kaluza software. Analysis was performed by FMO (fluorescence minus one) to discard nonspecific binding.

Activated lymphocytes (incubated with tumor and peptide) were subjected to the same procedure for FACS analysis.

### C. Protocol for the Identification of C24D Binding Receptor on Human PBMC.

Cell lysis: Fresh PBMCs (15×10⁶) previously washed with cold PBS, were lysed after 20 minutes on ice with buffer lysate (150 mM NaCl, 1% triton X-100, 50 mM Tris HCl (pH 8), to 100 ml with DDW with the addition of protease inhibitors).

The lysates were centrifuged (12,000 rpm, 4° C.) and supernatant samples were transferred to EPPENDORF™ tubes. A small volume of the lysate was removed to perform a protein quantification assay.

After addition of Laemmly sample buffer and boiling of the samples at 95° C. for 5 minutes, samples were stored at −80° C. until use.

C24D specific binding for protein identification: Precipitation of peptide binding specific protein for mass spectrometry was performed using streptavidin magnetic beads (μMACS streptavidin Kit, Miltenyi Biotec GmbH. Cat. Number 130-074-101). Cell protein (50 μg/200 μl lysis buffer) from fresh PBMC of healthy donors, according to the results obtained by FACS analysis, was incubated with 15 μg biotinylated peptide for 1 hour at 4° C. at constant shaking. Micro-magnetic beads were added for 5 minutes, following manufacturer instructions. The complex was placed in a micro-column in the magnetic field of a μMACS Separator. After rinsing the column, the target molecules which bound to the biotinylated probe were eluted and subjected to cell electrophoresis.

### Samples:

1. PBMC lysates obtained from donor A precipitated with biotinylated-C24D at the N terminus.
2. PBMC lysates obtained from donor A precipitated with C24D biotinylated at the C terminus.
3. PBMC lysates obtained from donor B precipitated with biotinylated-C24D at the N terminus.
4. PBMC lysates obtained from donor B precipitated with C24D biotinylated at the C terminus.
5,6. Control sample of PBMC A or B without the precipitation with the peptide and only with the addition of streptavidin magnetic beads.

Gel electrophoresis: Equal amounts of protein (50 μg) were loaded into the wells of the SDS-PAGE gel (10%), along with molecular weight marker. The gel ran for 1 hour at 150 V. The gels were stained with Imperial Protein Stain (Thermo Scientific, cat. Number 24615). Gels were sent for mass spectrometry analysis. Total: 6 samples.

In-gel proteolysis and mass spectrometry analysis: The proteins in the gel were reduced with 2.8 mM DTT (60° C. for 30 min), modified with 8.8 mM iodoacetamide in 100 mM ammonium bicarbonate (in the dark, room temperature for 30 min) and digested in 10% acetonitrile and 10 mM ammonium bicarbonate with modified trypsin (Promega) at a 1:10 enzyme-to-substrate ratio, overnight at 37° C. The tryptic peptides were desalted using C18 tips (Homemade stage tips) dried and re-suspended in 0.1% Formic acid.

The peptides were resolved by reverse-phase chromatography on 0.075×180-mm fused silica capillaries (J&W) packed with Reprosil reversed phase material (Dr Maisch GmbH, Germany). The peptides were eluted with linear 60 minutes gradient of 5 to 28% 15 minutes gradient of 28 to 95% and 15 minutes at 95% acetonitrile with 0.1% formic acid in water at flow rates of 0.15 μl/min. Mass spectrometry was performed by Q Exactive plus mass spectrometer (Thermo) in a positive mode using repetitively full MS scan followed by collision induces dissociation (HCD) of the 10 most dominant ions selected from the first MS scan.

The mass spectrometry data was analyzed using Proteome Discoverer 1.4 software with Sequest (Thermo) and Mascot (Matrix Science) algorithms against Human Uniprot database with 1% FDR. Semi quantitation was done by calculating the peak area of each peptide based its extracted ion currents (XICs), and the area of the protein is the average of the three most intense peptides from each protein.

Results

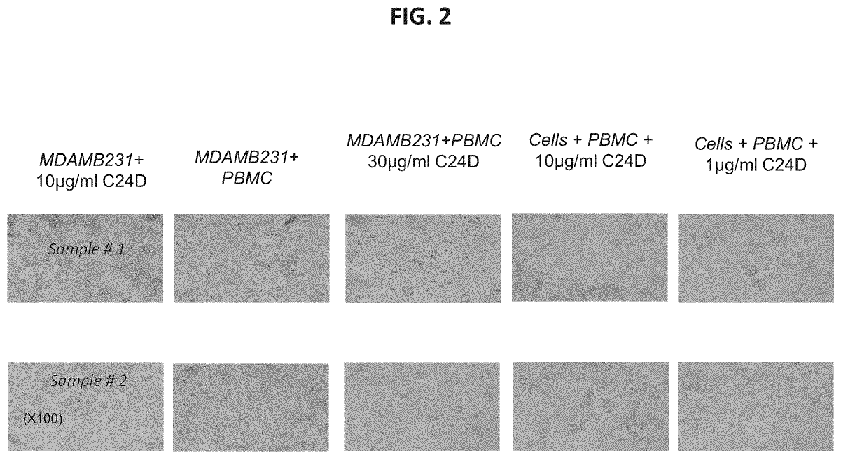

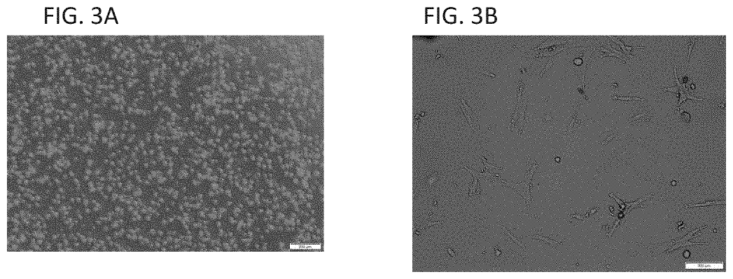

C24D binding to leucocytes reduced tumor cell density: To determine the optimal dose for inducing tumor cell cytotoxicity, different concentrations of C24D peptide were tested. Results demonstrated that C24D at 10 μg/ml induced tumor cell killing 4-6 days after tumor incubation with PBMC of healthy female donors in the following breast cancer cell lines: MCF7, MDAMB231 and MDAMB468 cells. In FIG. 2 it can be seen that C24D at 10 μg/ml peptide induced cell density reduction in tumor cells that varied from 50 to 80%, compared to tumor cells incubated only with PBMC.

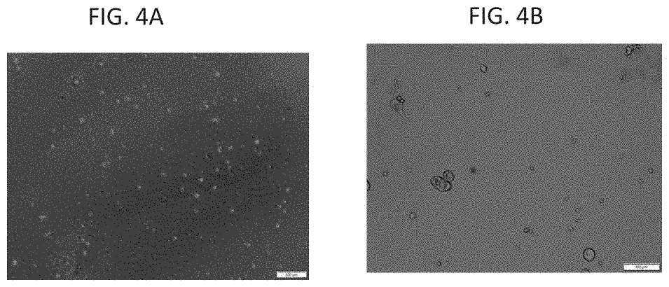

C24D binding to autologous leucocytes of breast cancer patients reduced the density of tumor cells derived from patient's biopsies, as seen in FIGS. 4A-B.

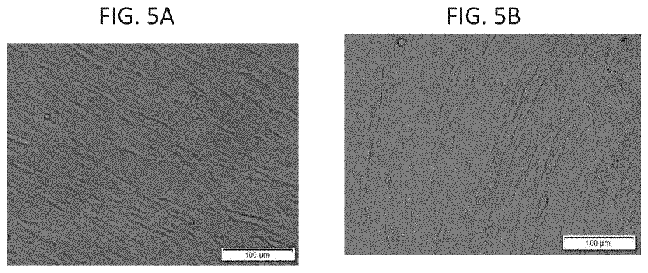

CD24D has no effect on normal stroma cells of the same patient as seen in FIGS. 5A-B.

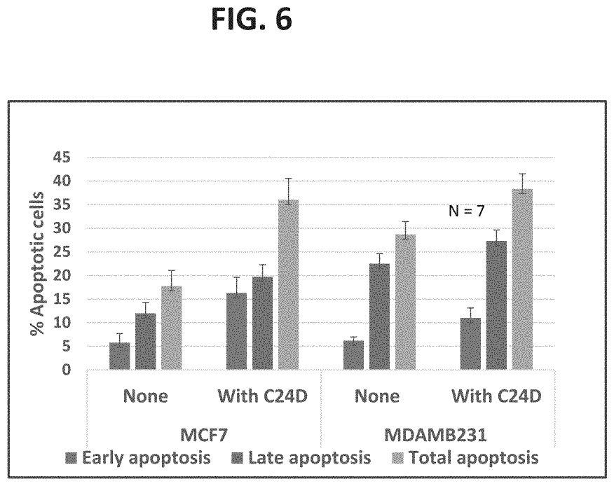

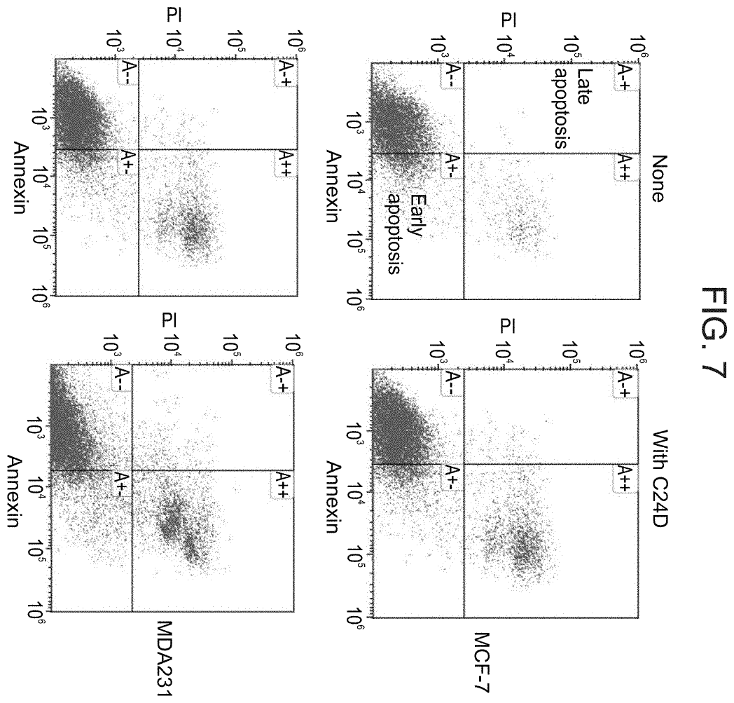

C24D treatment induced tumor cell apoptosis: Apoptosis was determined on days 4-6 in tumor adherent cells, after PBMC, with or without peptide addition, in 7 individual experiments. Adherent cells were those relatively few tumor cells that were still alive, or had already initiated the apoptotic process, after 4-6 days of incubation. The aim of this study was to demonstrate that C24D's induces the apoptotic pathway. As can be seen in FIGS. 6 and 7 and Tables 4 and 5, an apoptotic trend is perceptible in MCF7 cells treated with the peptide. In the aggressive, metastatic triple negative MDAMB231 cells, the induction of the apoptotic process was significant (p<0.05). Annexin V positive cells (early apoptosis) are the source of the significant differences obtained in the total of apoptotic cells.

TABLE 4

| Total Apoptosis | MCF7 | MDAMB468 | MDAMB231 |
|---|---|---|---|
| Without C24D | 17.95 ± 3.0 | 14.56 ± 2.8 | 7.52 ± 2 |
| With C24D | 23.48 ± 3.4 | 18.68 ± 3.1 | 13.53 ± 2.1 |

TABLE 5

| Early Apoptosis | MCF7 | MDAMB468 | MDAMB231 |
|---|---|---|---|
| Without C24D | 7.0 ± 1.8 | 8.2 ± 2.3 | 2.4 ± 0.9 |
| With C24D | 9.2 ± 2.3 | 10.1 ± 3 | 6.1 ± 1.5 |
| | | | p < 0.05 |

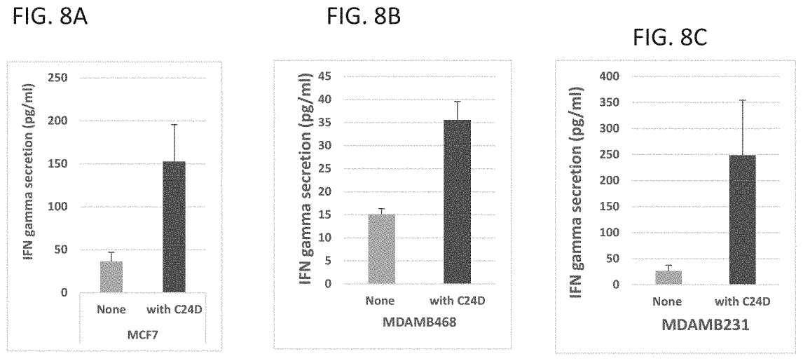

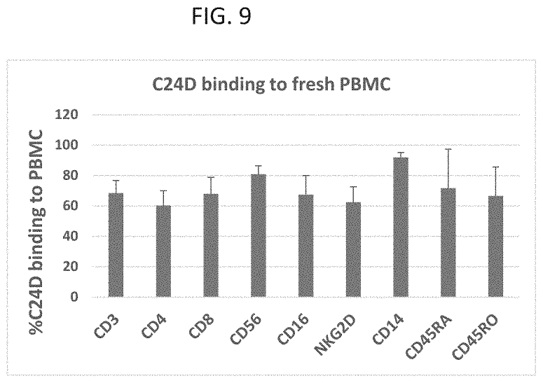

Peptide C24D induces IFNγ secretion: IFNγ secretion was measured in the supernatant of lymphocytes incubated with different tumor cells, with or without the addition of C24D for 4-6 days. Results demonstrated that C24D added to PBMC incubated on MDAMB231 increased IFNγ secretion by 9.7 fold. In experiments using MCF7 or MDAMB468, C24D induced 4.2 and 2.3-fold increase, respectively (FIGS. 8A-C).

C24D binding to fresh isolated PBMC: Studies of C24D binding to PBMC were performed with the FITC labeled C24D. Peptide binding in isolated mononuclear cells from eight female donors was analyzed. CD14, CD45RA and CD45RO were calculated only in two different PBMC. C24D bound to all PBMC sub-populations showing non-significant differences between the diverse leucocytes sub-populations.

C24D binds to activated lymphocytes sub-populations: Peptide binding to the diverse lymphocyte sub-populations exposed to tumor cells and incubated with C24D was evaluated. For this purpose, PBMC from healthy female donors was exposed to triple-negative and estrogen-positive tumor cell lines. FACS analysis was performed 4-6 days following cell incubation. The cells were also examined after short-term (2-3 days) incubation and no differences in the percent of C24D binding to cells compared to long-term incubation were noted.

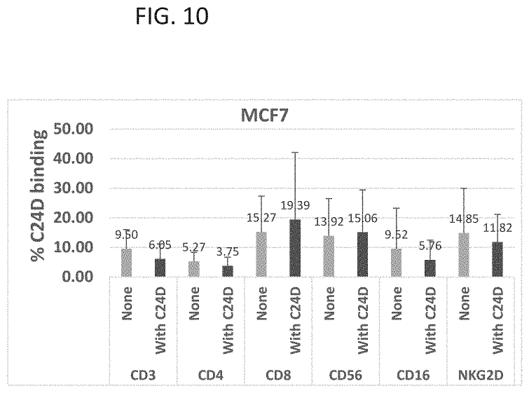

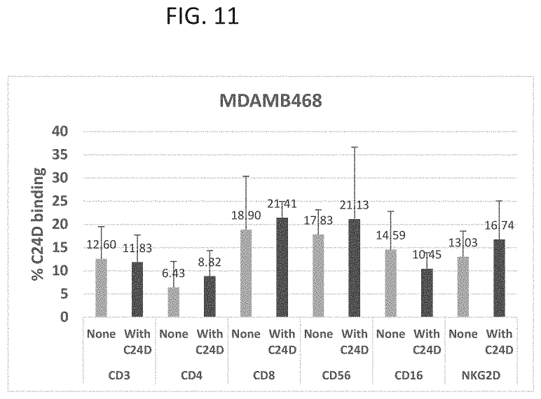

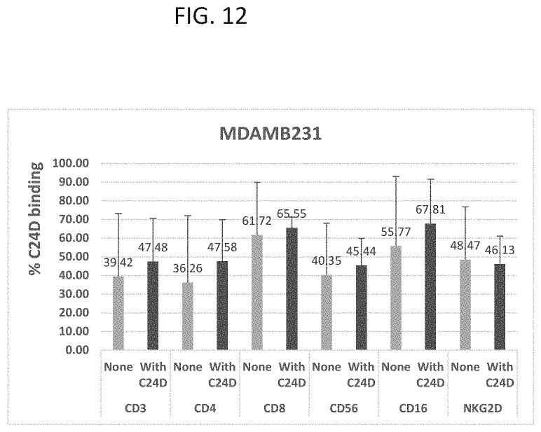

Results obtained with 5 different PBMC, isolated from 5 donors, showed that C24D binds T and NK cell sub-populations at different levels. The percent binding of lymphocyte sub-populations differs according to the activating tumor cells. PBMC incubated with MCF7 binds less peptide than the triple negative breast cancer cells (FIG. 10). The PBMC activated by the most aggressive triple negative cells (MDAMB231) demonstrated the greatest percent of peptide binding (FIG. 12).

No significant differences in binding were found with or without the presence of C24D in culture, likely because of the variations between the different donors.

CD8 T cells showed greater percent of peptide binding, compared to other T and NK cells.

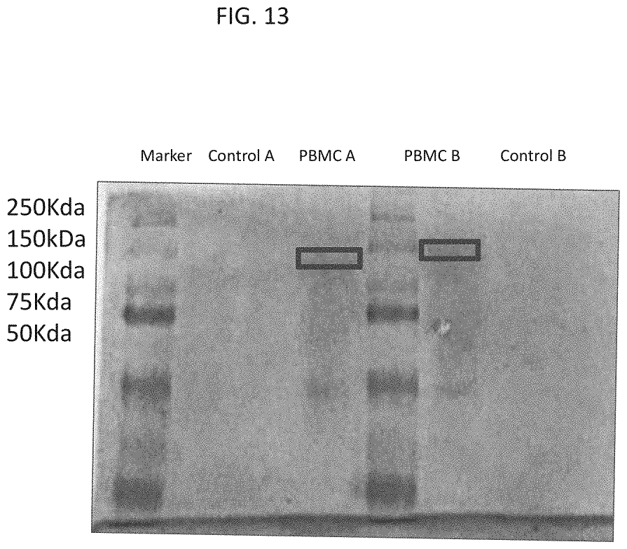

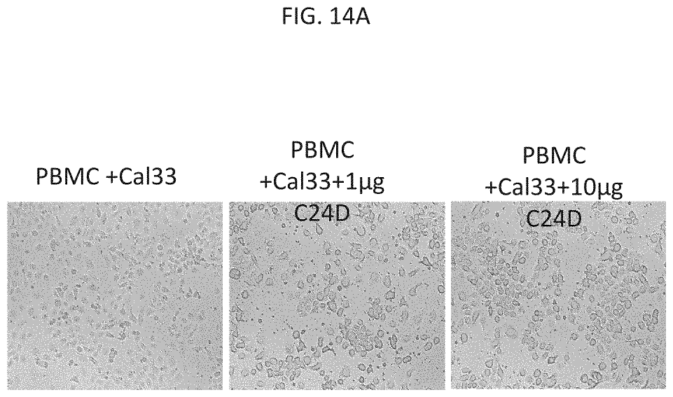

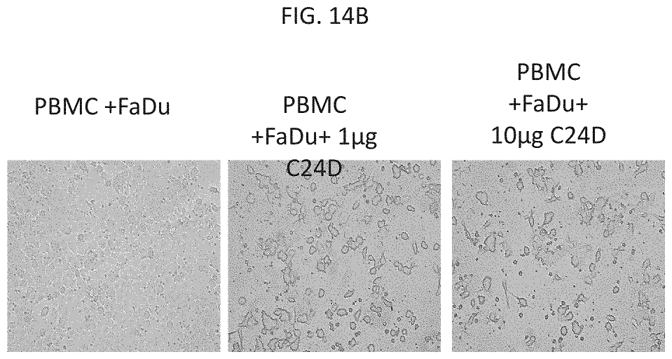

Identification of C24D binding receptor: For the identification of C24D binding receptor, initially, peptide precipitation of the C24D binding specific protein was performed. Then, precipitated PBMC lysates (50 μg/lane) samples were subjected to gel electrophoresis. For mass spectrometry analysis, 2 different PBMCs were analyzed: A and B and their respective controls as described in the material and methods. The precipitated complexes of lysate, biotinylated peptide and streptavidin magnetic beads were transferred to a column placed on a magnetic field and specific protein was eluted. The specific C24D binding protein was subjected to PAGE and the gel were sent for mass spectrometry analysis. 2 major lanes at approximately 140 Kda in each PBMC were observed—see FIG. 13.

Mass spectrometry analysis showed a unique cell surface receptor found in the two PBMC samples and not found in the control samples. The receptor identified was CD45 [X6R433, Receptor-type tyrosine-protein phosphatase C OS=Homo sapiens GN=PTPRC PE=1 SV=1−[X6R433_HUMAN.].

### Example 2

Materials and Methods

Head and Neck Cancer Cell Lines:

FADU: pharynx squamous carcinoma.

Cal 33: tongue squamous cell carcinoma

A 431: skin/epidermis epidermoid carcinoma

Cell Cultures:

Cal 33 and A431 human head and neck carcinoma cells were maintained in monolayer cultures in DMEM medium (Biological Industries, Israel) supplemented with 10% fetal bovine serum (FBS, Invitrogen, Gibco), 1% penicillin/streptomycin, and sodium 1 mM pyruvate (growth medium). FADU human head and neck carcinoma cells were maintained in monolayer cultures in MEM medium (Biological Industries, Israel) supplemented with 10% fetal bovine serum (FBS, Invitrogen, Gibco), 1% penicillin/streptomycin, and sodium 1 mM pyruvate (growth medium). For different cell passages, confluent monolayer cultures were trypsinized with trypsin 0.25%/EDTA 0.05% solution (Biological Industries), washed once in DMEM or MEM medium, and seeded in growth medium.

Isolation of PBMCs form leucocyte-enriched buffy coats: Blood from healthy female's donors were obtained from the Blood Bank. Blood was provided as buffy coats.

Peripheral blood mononuclear cells (PBMC) were isolated from buffy coats by density gradient centrifugation, using Ficoll-Paque Premium 1.073 (SIGMA).

After leucocyte isolation, the cells were re-suspended in medium RPMI+10% human AB serum for in-vitro experiments.

Protocol for the Assessment of C24D Activity:

Day 1: 4×10⁵ tumor cells from the cell line were incubated in the respective growth medium in 6 well plates overnight.

Day 2: 3 hours before PBMC of healthy female donors for the tumor cell lines addition, DMEM or MEM medium was changed to RPMI medium. PBMC were added on the tumor cells (2×10⁶/ml) and C24D was immediately added at 1 or 10 μg/ml and incubated for 24 or 48 hours, at 37° C., 5% CO₂.

Day 3: At the end of the experiments, lymphocytes were extracted, centrifuged and re-suspended in PBS supplemented with 0.1% Na Azide and 5% FBS for FACS analysis. Supernatant recovered from lymphocytes centrifugation was stored at −80° C. for interferon gamma determination. Tumor cells were washed for tumor cell density assessment.

C24D activity was evaluated by tumor cell density and interferon gamma secretion.

Tumor cell density: Tumor cells were washed once with PBS and cell density was analyzed by inverted microscopy (image magnification ×10).

Interferon gamma secretion: Supernatants obtained 3 days after tumor cell incubation with lymphocytes and peptides were used for interferon gamma secretion using: Human Interferon gamma ELISA Ready SET-Go. eBioscience, according to manufacturer instructions.

FACS analysis of activated PBMC sub-populations by C24D: Fresh isolated PBMC from healthy donors incubated on A 431 head and neck carcinoma cells for 48 hours as described above, were incubated with the following antibodies: CD4-PC7, CD8-KO, CD56-PE, NKG2D-APC, CD14-KO, CD45RO-PC5.5 and activation markers CD57-FITC, and CD69-PC5.5 (Beckman Coulter) for 40 minutes at room temperature. Subsequently, cells were washed twice (10 min. 1200 rpm, 4° C.). Gated live cells were analyzed using Coulter Navious FACS, Kaluza software. The percentage of the following sub-populations were analyzed by flow cytometry analysis: CD4/CD69, CD8/CD69, CD56/CD69, CD56/CD57 and NKG2D/CD57. PBMCs from cultures without the addition of the peptide were used as controls.

Results

C24D Binding to Leucocytes Reduced Tumor Cell Density:

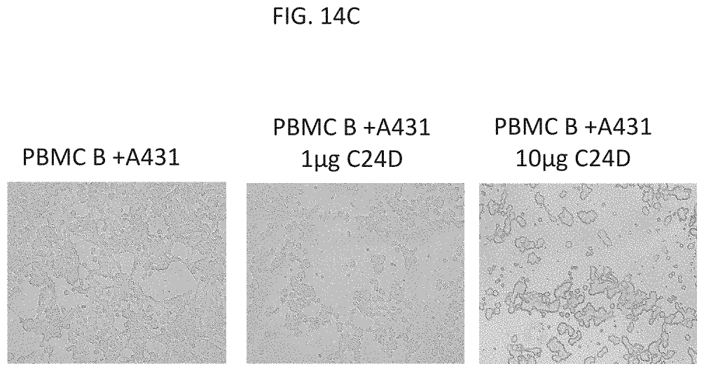

Results demonstrated that C24D at 1 or 10 μg/ml induced tumor cell killing 24-48 hours after tumor incubation with PBMC of healthy donors in the following head and neck cancer cell lines: FADU, Cal 33 and A 431 cells. In FIGS. 14A-C, it can be seen that C24D at 10 μg/ml peptide induced cell density reduction in tumor cells that varied from 50 to 70%, compared to tumor cells incubated only with PBMC.

Peptide C24D Induces IFNγ Secretion:

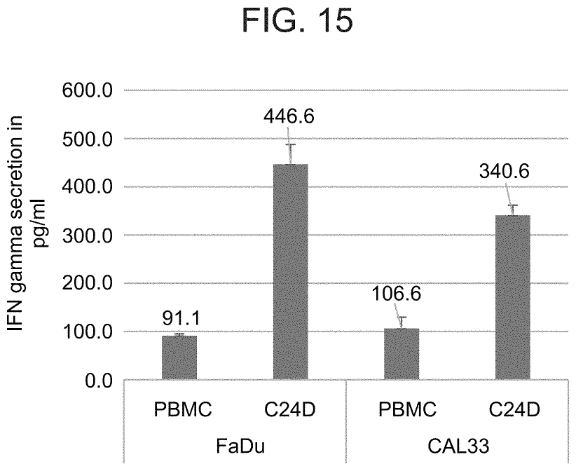

IFNγ secretion was measured in the supernatant of lymphocytes incubated with Cal33 and FADU cells tumor cells and with or without the addition of C24D (10 μg/ml) for 2-3 days. Results demonstrate that C24D added to PBMC incubated on the different head and neck tumor cells increased IFNγ secretion (FIG. 15).

Peptide C24D Induces Activated PBMC Sub-Populations:

Addition of 10 μg/ml C24D peptide to PBMC from healthy donors co-cultured with A431 head and neck tumor cells induced the activation of T and NK cells. The activation of T (CD8+ cells) and NK (CD56+ cells) cells was evaluated by FACS analysis.

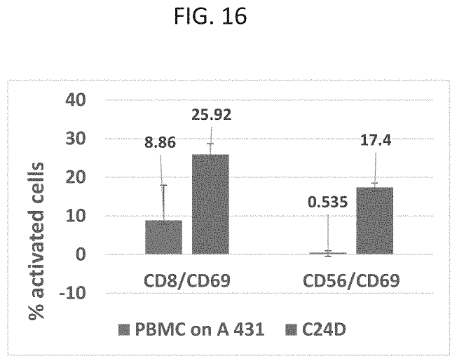

The activation of T lymphocytes and Natural Killer (NK) cells, both in vivo and in vitro, induces expression of CD69. This molecule, which appears to be the earliest inducible cell surface glycoprotein acquired during lymphoid activation, is involved in lymphocyte proliferation and functions as a signal-transmitting receptor in lymphocytes and natural NK cells. FIG. 16 shows that the induction of C24D expression caused an increase in the percentage of CD8+/CD69+ cells and CD56+/CD69+ cells in two different experiments.

### Example 3

Materials and Methods:

Blocking of CD45 receptor: The antibody anti-CD45: Abcam 10 μg/ml (catalog number: ab123522) or the peptide CD45 inhibitor VI (Calbiochem, catalog number: 5.30197.0001) were used for CD45 blocking. The blockers were added to PBMCs prior to the addition of PBMCs to the tumor plates.

CD45 Signal Transduction Determination:

Day 1: 2.5×10⁵ tumor cells/ml (derived from an exponentially growing monolayer) were incubated in DMEM+10% FBS in 6 well plates overnight (500,000 cells per well).

Day 2: 3 hours prior to PBMC addition, DMEM medium was changed to complete RPMI in the 6 well plates incubated with tumor cells. PBMCs were added to the tumor cells (2×10⁶/ml) and immediately C24D was added at 10 μg/ml and incubated for 5, 15, 30, 60 minutes and 24 hours at 37° C., 5% CO₂.

At the end of each allotted incubation period, lymphocytes were extracted, centrifuged and re-suspended in 0.12 ml of lysis buffer (20 mM Tris-HCl pH 7.5, 150 mM NaCl, 1 mM NaF, 2 mM Na₃VO₄, 1% NP40, 10 mM b-glycerophosphate, 30% glycerol, 1 mM EDTA, 0.5% sodium-deoxycholate, 0.5% protease inhibitor cocktail), followed by one freeze-thaw cycle for 20 min. Cells were harvested and centrifuged (14,000 rpm, 15 min, 4° C.). The supernatants were collected, and aliquots were separated on 10% SDS PAGE, followed by Western blotting with anti-phospho-Lck Y505 (0.5 μg/ml, ab4901, Abcam) and anti-phospho-ZAP70 Y493 (1 μg/ml, ab194800, Abcam). GAPDH (1 μg/ml, ab9485, Abcam) was added as a control for sample loading. An Odyssey infrared scanner (LI-COR) was used for detection.

Percentage (%) of maximal phosphorylation (p-Lck Y505 or p-ZAP70Y493) were first normalized to the levels obtained with GAPDH respectively, and the activation values were normalized for each treatment vs. its control (e.g. C24D+Lymphocytes vs. Lymphocytes control (without C24D). Then, the values obtained were expressed as % of maximal activation that was observed at each time point in each experiment.

Western Blot Analysis:
a. Determination of protein concentration in each sample: 40 μg
b. Gel preparation (10%) and number of wells (10): 40% Acrylamide (01-876-1A, Biological Industries Israel Beit-Haemek), lower buffer (TRIS8815-500 ml, Tivan Biotech), upper buffer (TRIS681500, TivanBiotech), Transfer (TG10500 ml, BioPrep), Running (TGS10-500 ml), Temed (805613, MP Biomedicals LLC), ammonium persulfate (A-3678, Sigma), SDS (419300163, Bioworld).
c. Samples were separated on 10% SDS PAGE for 1.5 h, 140V and protein ladder (PM007-0500, GeneDirex) was loaded in the first lane.
d. Proteins were then transferred into nitrocellulose membrane (88018, Thermo scientific) for 1.5 h, 100V.
e. At the end of the transfer time, the membrane was blocked in 5% BSA (220700082, Bio world) and TBST (TBSVE3-500 ml, BioPrep) for 1 h.
f. At the end of the blocking time, the membrane was washed three times with TBS-T (0.1%), each wash for 10 minutes.
g. The membrane was incubated with the primary antibody, p-LCK Y505 or p-zap70 Y493+GAPDH overnight in 4° C.).
h. The primary antibody was collected, and the membrane was washed three times with TBS-T (each wash for 10 min).
i. The secondary antibody, IRDye 800CW Goat anti-Rabbit (1 mg/ml, 926-32211, LI-COR) was added for 1 h.
j. At the end of the incubation time, the membrane was washed three times with TBS-T (each wash for 10 min).

Quantification methods: An Odyssey infrared scanner (LI-COR) was used for detection and quantification and the phosphorylation was quantitated by Image J (NIH, USA). Percentages (%) of maximal phosphorylation (p-LCK505 or p-ZAP70) were first normalized to the levels obtained with GAPDH respectively, and the activation values were normalized for each time point vs. its control, without C24D (e.g. C24D+Lymphocytes vs. Lymphocytes control). Then the values obtained were expressed as % of maximal activation that was observed in each experiment at each time point.

TABLE 6
Gels-2

| Lower-10% | Upper- 5% |
|---|---|
| ddH20 - 8 ml | ddH20 - 3.9 ml |
| 40% Acrylamide- 4 ml | 40% Acrylamide- 0.8 ml |
| 1.5M Tris-HCl pH 8.8 - 4 ml | 1.5M Tris-HCl pH 6.8 - 1.55 ml |
| 20% SDS - 80 μl | 20% SDS - 40 μl |
| APS 10% - 110 μl | APS 10% - 60 μl |
| Temed- 11 μl | Temed- 6 μl |

Interferon gamma secretion: Supernatants obtained 4-6 days after tumor cell incubation with lymphocytes and peptides or blocked PBMCs were used for interferon gamma secretion using: Human Interferon gamma ELISA Ready SET-Go. eBioscience, cat. Number: 88-7316-22, according to manufacturer instructions.

Results

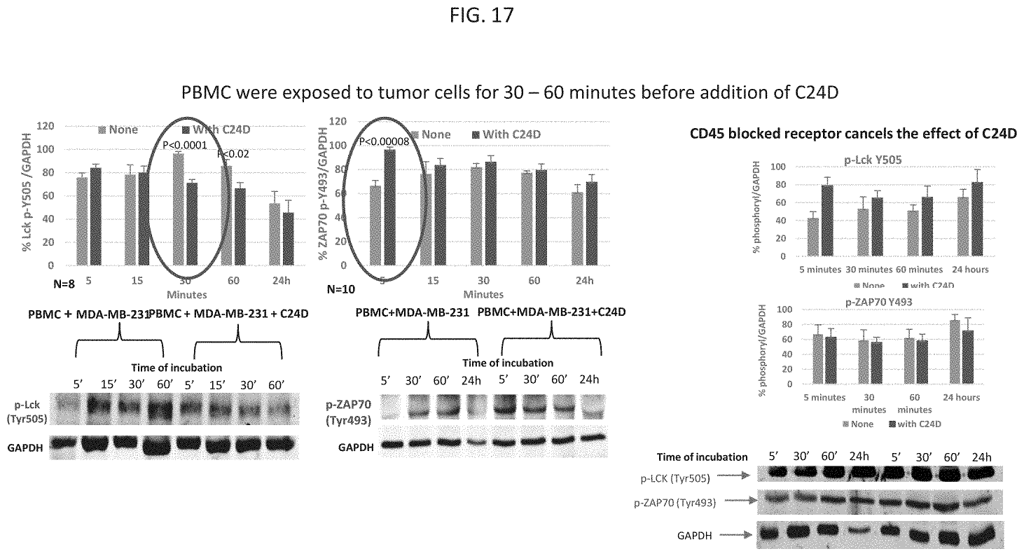

Addition of C24D to the tumor cells and PBMCs for 30 and 60 minutes reduced Lck Y505 phosphorylation significantly (p<0.0001 and p<0.02 respectively). Lck (or lymphocyte-specific protein tyrosine kinase) phosphorylates tyrosine residues of certain proteins involved in the intracellular signaling pathways of these lymphocytes. It is a member of the Src family of tyrosine kinases (1). The de-phosphorylation of the Y505 in Lck results in Lck increase in catalytic activity (2). Lck activation induce in turn, the activation of Zap70 by significant phosphorylation of the Y493 in Zap70 (p<0.0008), a member of the protein-tyrosine kinase family. Zap70 is a protein normally expressed near the surface membrane of T cells and natural killer cells. It is part of the T cell receptor and plays a critical role in T-cell signaling (3) (FIG. 17).

Blocking of CD45 with the above described blockers reduced C24D Lck and Zap70 activation, confirming C24D binding to CD45 (FIG. 17).

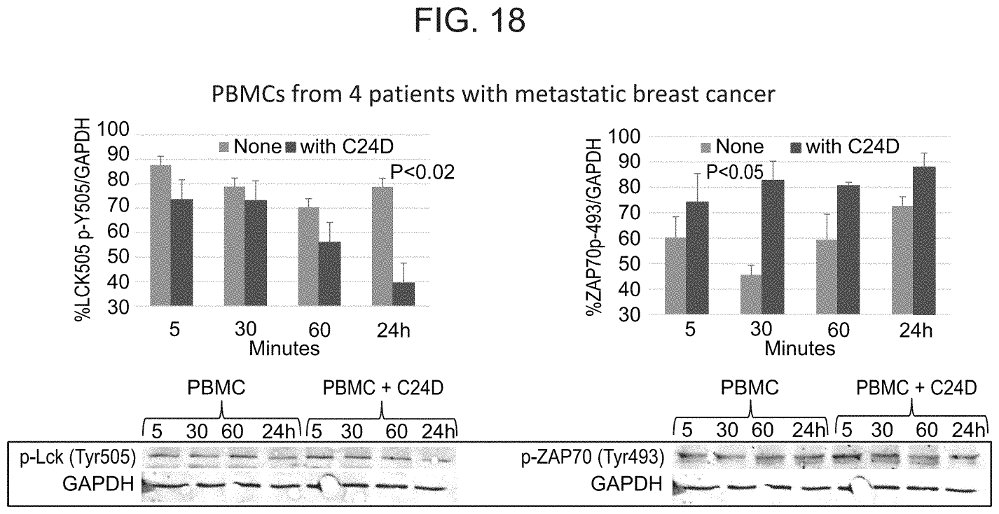

PBMCs of metastatic breast cancer patients were lysed following incubation with C24D for different times. C24D induced significant Lck Y505 de-phosphorylation (p<0.02). As a result, Zap70 Y493 was significantly phosphorylated (p<0.05) 5 to 30 minutes after peptide addition (FIG. 18).

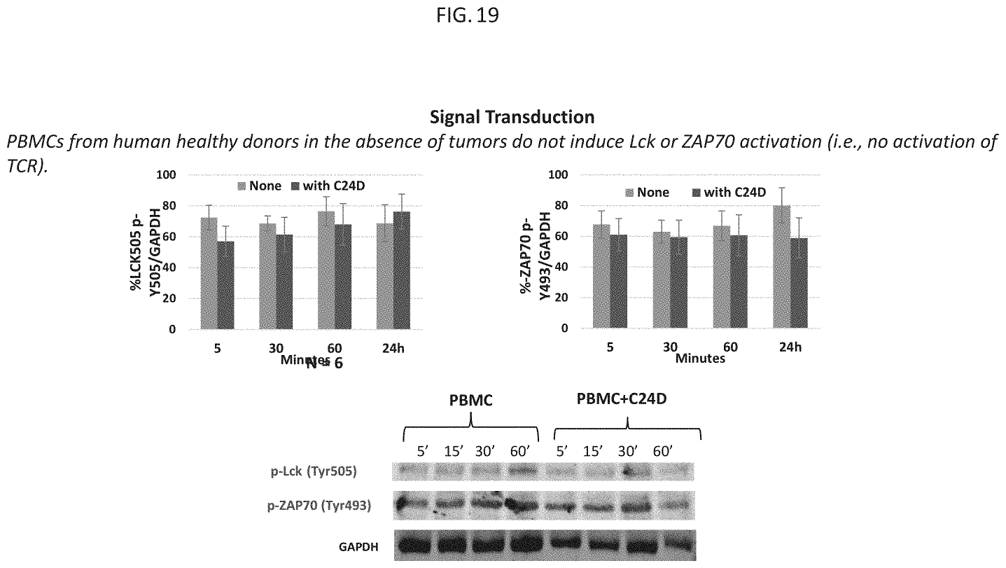

In contrast to the PBMCs of breast cancer patients, C24D failed to activate Lck and Zap70 in PBMCs from healthy donors (FIG. 19).

FIG. 20 illustrates that blocking of CD45 receptor prevents C24D binding, resulting in inhibition of IFNγ secretion, confirming that C24D binds CD45 on PBMCs to activate lymphocytes.

Conclusions: In this study a new mechanism of tumor immune suppression based on tumor inhibition of Lck and ZAP70 in T and NK cells is described. By binding to CD45 on leukocytes previously exposed to tumor cells, C24D reverses tumor-induced immune-suppression, resulting in tumor cell killing.

### REFERENCES FOR EXAMPLE 3

1. Shaun-Paul Cordoba, Kaushik Choudhuri, Hao Zhang, Marcus Bridge, Alp Bugra Basat, Michael L. Dustin, and P. Anton van der Merwe The large ectodomains of CD45 and CD148 regulate their segregation from and inhibition of ligated T-cell receptor. Blood. 2013; 121(21):4295-4302.
2. Dominik Filipp, Ondrej Ballek and Jasper Manning. Lck, membrane microdomains, and TCR triggering machinery: defining the new rules of engagement. Frontiers in immunology (3):155, 2012.
3. Leah V. Sibener, Ricardo A. Fernandes, Elizabeth M. Kolawole, Catherine B. Carbone, Fan Liu, Darren McAffee, Michael E. Birnbaum, Xinbo Yang, Laura F. Su, Wong Yu, Shen Dong, Marvin H. Gee, Kevin M. Jude, Mark M. Davis, Jay T. Groves, William A. Goddard III, James R. Heath, Brian D. Evavold, Ronald D. Vale and K. Christopher Garcia. Isolation of a Structural Mechanism for Uncoupling T Cell Receptor Signaling from Peptide-MHC Binding. Cell 174, 672-687, Jul. 26, 2018.

### Example 4

Materials and Methods:

Human cell lines and culture conditions: MDA-MB-231 (obtained from ATCC, Biological Industries, Kibbutz Beit Ha Emek, Israel) is a triple negative breast cancer cell line that is Her2-neu, estrogen receptor (ER)- and progesterone receptor (PR)-negative. MCF-10A (obtained from ATCC) are normal breast cells. Cells were grown in DMEM supplemented with 10% FCS, L-glutamine (2 mM), Na-pyruvate (1 nM), penicillin (100μ/ml), streptomycin sulfate (0.1 mg/ml) and nystatine (12.5μ/ml) (complete culture medium) (Biological Industries) and cultures were maintained at 37° C. in a humidified 5% CO₂ incubator. A large stock of cells was prepared to maintain homogeneity and tumorigenicity of the cell line. Cells were not used beyond passage five and examination for mycoplasma (Mycoplasma detection kit, Biological Industries) at least once every six months.

PBMC-Tumor Cell Co-Cultures and Peptide Efficacy:

PBMC isolation: Peripheral blood from healthy female donors or breast cancer patients were isolated from blood. Samples were isolated by Ficoll-Hypaque density gradient (d=1,077 g/mL, Ficoll-Paque Plus, GE Healthcare, Upsalla, Sweden) by centrifugation at 650×g for 30 minutes.

Tumor cells (4×10⁵ derived from an exponentially growing monolayer) were incubated in complete medium overnight in 6 well plates. PBMC in complete RPMI medium supplemented with 5% AB serum instead of FBS (Biological Industries) were added on the tumor cells (2×10⁶/ml) and C24D peptide was added immediately at 10 μg/ml and incubated for different times at 37° C., 5% CO₂.

CD45 Signal Transduction Determination:

PBMCs were incubated with the tumor cells (2×10⁶/ml) and the peptide was added immediately at 10 μg/ml and incubated for 5, 15, 30, 60 minutes and 24 hours at 37° C., 5% CO₂.

At the end of each allotted incubation period, lymphocytes were extracted, centrifuged and re-suspended in 0.12 ml of lysis buffer (20 mM Tris-HCl pH 7.5, 150 mM NaCl, 1 mM NaF, 2 mM Na₃VO₄, 1% NP40, 10 mM b-glycerophosphate, 30% glycerol, 1 mM EDTA, 0.5% sodium-deoxycholate, 0.5% protease inhibitor cocktail), followed by one freeze-thaw cycle for 20 min. Cells were harvested and centrifuged (14,000 rpm, 15 min, 4° C.). The supernatants were collected, and aliquots were separated on 10% SDS PAGE, followed by Western blotting with anti-phospho-Lck Y505 (0.5 μg/ml, ab4901, Abcam, Cambridge, UK), anti-phospho-Lck Y394 (1 μg/ml, ab201567, Abcam), anti-phospho-ZAP70 Y493 (1 μg/ml, ab194800, Abcam) and anti-phospho-VAV1 (2 μg/ml, ab76225, Abcam). GAPDH (1 μg/ml, ab9485, Abcam) was added as a control for sample loading. After several washings, the secondary antibody, IRDye 800CW Goat anti-Rabbit or (1 mg/ml, 926-32211, LI-COR, Nebraska, USA) was added for 1 h.

Quantification methods: The membrane was analyzed by Odyssey 2.1 (Infrared Imaging System) for specific band identification. Quantification of phosphorylation was done by Image J (NIH, USA). Percentage (%) of maximal phosphorylation was first normalized to the levels obtained with GAPDH respectively, and the activation values were normalized for each time point vs its control, without C24D (e.g., C24D+Lymphocytes vs. Lymphocytes control). The values obtained were expressed as % of maximal activation that was observed in each experiment, in each time point.

Results

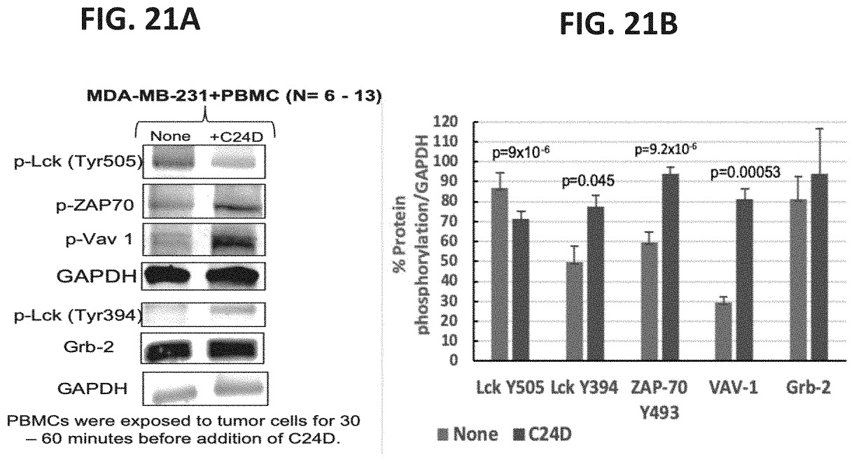

MDA-MB-231 were co-cultured with PBMCs from healthy female donors for 30 minutes. C24D was then added to cultures for different times. Addition of C24D to cultures reversed tumor-induced immunosuppression. As illustrated in FIGS. 21A-B, the percentage of protein phosphorylation after addition of C24D for 30 minutes decreased significantly from 86.7±1.8 to 72.2±2.9 in the Y505 of Lck (p=9×10⁻⁶). At the same time, the phosphorylation of the Y394 in the Lck increased from 49.7±8.1 to 77.3±5.7 (p=0.045). Five minutes after addition of peptide to cells, the Y493 in Zap70 increased from 59.5±5.3 to 94.2±3.2 (p=9×10⁻⁶). The phosphorylation of VAV1 increased 29.3±3.1 to 80.9±5.2 (p=0.00053), 30 minutes after peptide addition. These results indicate activation of the TCR in lymphocytes.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

In addition, any priority documents of this application are hereby incorporated herein by reference in their entirety.

1. A method of treating a disease selected from the group consisting of an autoimmune disease, a neurodegenerative disease and an infectious disease in a subject in need thereof, the method comprising administering to the subject a therapeutically effective amount of a multimeric peptide comprising at least two peptide monomers linked to one another, each of said at least two peptide monomers comprising at least 6 consecutive amino acids from the amino acid sequence as set forth in SEQ ID NO: 1, wherein said at least two peptide monomers are each no longer than 30 amino acids, wherein said multimeric peptide binds to Receptor type tyrosine-protein phosphatase C (CD45), with the proviso that the infectious disease is not a retrovirally-mediated disease, thereby treating the disease.

2. (canceled)

3. The method of claim 1, wherein the peptide is capable of increasing INF-γ secretion from activated leukocytes.

4. The method of claim 1, wherein the peptide is a dimer.

5. The method of claim 1, wherein each of said at least two peptide monomers comprise no more than 15 consecutive amino acids from the amino acid sequence as set forth in SEQ ID NO: 1.

6. The method of claim 1, wherein said at least two peptide monomers comprise an identical amino acid sequence.

7. The method of claim 1, wherein each of said at least two peptide monomers is attached to a Cysteine (Cys) residue.

8. The method of claim 7, wherein the carboxy end of said at least two peptide monomers is attached to said Cys residue.

9. (canceled)

10. The method of claim 7, wherein said at least two peptide monomers are linked to one another by a disulfide bond.

11-12. (canceled)

13. The method of claim 1, wherein each of said two at least two peptide monomers comprise the sequence selected from the group consisting of SEQ ID NOs: 2-7.

14. The method of claim 1, wherein each of said at least two peptide monomers consists of the sequence selected from the group consisting of SEQ ID NOs: 8-13 and 101.

15. (canceled)

16. The method of claim 1, wherein said multimeric peptide consists of the sequence as set forth in SEQ ID NO: 102.

17-37. (canceled)

38. A method of treating triple negative breast cancer or head and neck cancer in a subject in need thereof, the method comprising administering to the subject a therapeutically effective amount of an agent that binds to Receptor type tyrosine-protein phosphatase C (CD45), thereby treating the triple negative breast cancer or the head and neck cancer.

39. (canceled)

40. The method of claim 38, wherein said agent is a peptide.

41. The method of claim 40, wherein said peptide is a multimeric peptide comprising at least two peptide monomers linked to one another, each of said at least two peptide monomers comprising at least 6 consecutive amino acids from the amino acid sequence as set forth in SEQ ID NO: 1, wherein said at least two peptide monomers are each no longer than 30 amino acids.

42. (canceled)

43. The method of claim 40, wherein the peptide is a dimer.

44. (canceled)

45. The method of claim 41, wherein each of said two at least two peptide monomers comprise the sequence selected from the group consisting of SEQ ID NOs: 2-7.

46. The method of claim 41, wherein each of said at least two peptide monomers consists of the sequence selected from the group consisting of SEQ ID NOs: 8-13 and 101.

47. The method of claim 41, wherein said at least two peptide monomers are covalently linked to one another.

48. (canceled)

49. A method of monitoring the efficacy of a therapeutic agent that increases the cytotoxicity of T cells by binding to CD45 in a subject, the method comprising analyzing in the T cells of the subject the phosphorylation status of at least one protein selected from the group consisting of Lck, ZAP70 and VAV-1, wherein:
(i) a decrease in the phosphorylation status of lymphocyte-specific protein tyrosine kinase (Lck) at position 505 is indicative of an efficacious therapeutic agent;
(ii) an increase in the phosphorylation status of Lck at position 394 is indicative of an efficacious therapeutic agent;
(iii) an increase in the phosphorylation status of Vav Guanine Nucleotide Exchange Factor 1 (VAV-1) is indicative of an efficacious therapeutic agent; and/or
(iv) an increase in the phosphorylation status of Zeta-chain-associated protein kinase 70 (ZAP-70) at position 493 is indicative of an efficacious therapeutic agent.

50. The method of claim 49, wherein the agent is a multimeric peptide comprising at least two peptide monomers linked to one another, each of said at least two peptide monomers comprising at least 6 consecutive amino acids from the amino acid sequence as set forth in SEQ ID NO: 1, wherein said at least two peptide monomers are each no longer than 30 amino acids.
